SEQUENCE LISTING (24 sequences)

SEQ ID NO: 1 - DNA, 1200 bp, Artificial Sequence
  Synthetic
atggagttatcactctcatcatcttcttcttcatcccttccccaacttcatactcatccttcatcatcatcatcttcttcacattacataaaaaaatcacctttttttattaataaattcaataatcacaccaaatgcaaattccacaattcctctgctctgagaactaatttcttctacactaccataactaaaacctcatcatcaagattcgttctaaacaaaaacccaaaccaattttccgtcaaggcttgcagtcaagttggttctgctggatccgatccagcattgaataaagttgcagactttaaagatgcattttggaggtttctaaggccccatactattcgtgggacagcattaggatcagtgtctttagtaacgagagcactacttgaaaacccaaacttgattcggtggtcacttttgctcaaggcattttcaggtcttgttgctttgatatgtgggaatggttatatagtcgggatcaatcagatctatgatatcggtattgataaggtgaacaaaccatatttacctattgctgcgggagatctttctgtccagtcagcatggtttttggtgttagcatttgcaatggtaggcgttattattgttgggatgaacttcggcccattcatcacctccctttattctctcggtcttttcttgggcaccatctattccgttccaccacttcgaatgaagagatttcctgttgttgcatttcttatcatcgccacggtgagaggttttcttctaaattttggtgtgtattatgcggttagagcagctctgggactaacattccaatggagctcagcagtggcttttatcacaaccttcgttacattatttgctttagtcattgccattactaaagatcttcctgatgtagagggtgaccgaaagtttcaaatttctacttttgcaacaaaacttggagtaagaaacattgcattattagggtcaggacttctgctgatcaattatattgggtctatcgttgcagcactttacatgcctcaggctttcaggagcagcttgatgataccattacataccatattagcttcctgtttgatttaccaggcatggatacttgagcgtgcgaattacacccaggaggcgatagctgggtactaccgatttgtatggaatctgttttattcagagtacatcatatttcctttcatctga

SEQ ID NO: 2 - DNA, 1119 bp, Artificial Sequence
  Synthetic
atggctactatggcttcttctttgctgaatcctctttcttgttccattaaacccaactcaaacagactaccattaccaacacccatttctctatctcgttcttgtagaaggctaacaatcaaagcaacggagacagatgcaaatgaagtgaagccaaaggcgccagagaaagcaccagctgcaagtggatctggttttaatcaaattcttgggattaaaggggctaaacaagaaactaataaatggaagatccgtgttcaacttacaaagccggttacttggcctccattaatttggggagtcgtatgtggagctgctgcttctggtaacttccaatggactgtggaagatgttgctaaatcaattgtttgcatgttgatgtctggcccatttctaaccggttacacacagacgatcaatgattggtatgatagagacattgatgctattaatgaaccttaccgtccaattccttccggagccatatctgaaaatgaggtcattactcaaatttgggtacttcttttaggaggcatcggattggctggtatattagacgtgtgggcagggcataagtcccctacaatattctatcttgctttgggtggatcattgttatcttatatctactcagctccacctttaaagctcaaacagaatggatggattggcaactttgcattaggagcaagctatattagcttaccatggtgggctggtcaagcattgttcggaactcttacacctgatatagtagttctcacacttttgtacagcatagctgggcttggtattgctatagtaaatgactttaaaagtgttgaaggagacaggaaaatggggcttcagtcccttcccgtggcttttggtgaagagacagctaaatggatatgtgttggtgccattgacataactcaactctctattgcaggttaccttttaggatctggtaaaccatattacgccttagcactcgttgggttgattgttccacaaatcttttttcagttcaagtactttcttaaagatccagttaaatatgatgtcaagtatcaggctagtgctcaaccatttctcattcttggtcttctggtgactgccttagctactagtcactga

SEQ ID NO: 3 - DNA, 1188 bp, Artificial Sequence
  Synthetic
atgaagtctttgattattgggtctttttctaataaggtttcttgttattccccatcattaccagattcatcttcttcacttataccaacaggttgttatcatgtatcactaagaacatttcagcgtaaccgagccattcaagctcaatcaagtcttgtgagatgcaatattggcaaattcaatgaaacattactactttcgcggaaacgaagtacaaaacatgttgcatgtgcggtttctgaacaacccattgaaccagatgctacaaaccctcaaagttcattaccaaatgctttggatgctttctataggttttcaagacctcatacagttataggaactgcattgagcatagtttcggtttcactcctagcggttcaaaagctttcggatttttctccactattcttcattggcgttttcgaggctattgttgctgccttctttatgaacatatacattgttggcttgaaccagctatccgatattgaaatagacaaggttaacaagccgtaccttccattggcatctggagaatattcagttcaaactggtattatcattgtatcatcatttgcagtcatgagtttctggcttggatggatcgtgggctcatggcctttattttgggcacttttcataagttttcttctagggaccgcatattcaatcaatataccgatgttgagatggaagcgctttgctcttgtggcagcaatgtgtattctagctgtaagagctattatagttcaagttgcattttatttgcacattcagacttttgtgtatggaagactcgccgtgttcccaaaacccgtgatatttgcaaccggatttatgagtttcttctctgttgttatagcattgttcaaggacatacccgacattgttggagacaagatttttggcattcaatcatttactgtccgtatgggtcaaaaacgggtgttttggatttgcatcttattacttgaaatagcttatggtgttgctattctagttggggcatcatctcccttcctttggagccgatacataacggtattgggtcatgcgattcttggtctgattctctggggtcgtgccaagtcaacggatctggagagcaaatcagcaataacctcattttacatgttcatatggcagttgttctatgccgagtatttgctcataccgctcgtgagatga

SEQ ID NO: 4 - DNA, 1167 bp, Artificial Sequence
  Synthetic
atggagttatcactctcatcatcttcttcttcatcccttccccaacttcatactcatccttcatcatcatcatcttcttcacattacataaaaaaatcacctttttttattaataaattcaataatcacaccaaatgcaaattccacaattcctctgctctgagaactaatttcttctacactaccataactaaaacctcatcatcaagattcgttctaaacaaaaacccaaaccaattttccgtcaaggcttgcagtcaagttggttctgctggatccgatccagcattgaataaagttgcagactttaaagatgcattttggaggtttctaaggccccatactattcgtgggacagcattaggatcagtgtctttagtaacgagagcactacttgaaaacccaaacttgattcggtggtcacttttgctcaaggcattttcaggtcttgttgctttgatatgtgggaatggttatatagtcgggatcaatcagatctatgatatcggtattgataaggtgaacaaaccatatttacctattgctgcgggagatctttctgtccagtcagcatggtttttggtgttagcatttgcaatggtaggcgttattattgttgggatgaacttcggcccattcatcacctccctttattctctcggtcttttcttgggcaccatctattccgttccaccacttcgaatgaagagatttcctgttgttgcatttcttatcatcgccacggtgagaggttttcttctaaattttggtgtgtattatgcggttagagcagctctgggactaacattccaatggagctcagcagtggcttttatcacaaccttcgttacattatttgctttagtcattgccattactaaagatcttcctgatgtagagggtgaccgaaagtttcaaatttctacttttgcaacaaaacttggagtaagaaacattgcattattagggtcaggacttctgctgatcaattatattgggtctatcgttgcagcactttacatgcctcaggctttcaggagcagcttgatgataccattacataccatattagcttcctgtttgatttaccaggcatggatacttgagcgtgcgaattacacccagcgatcacagtactttgacatgtcatcttgcaggaggcgatag

SEQ ID NO: 5 - DNA, 1116 bp, Artificial Sequence
  Synthetic
atggagttatcactctcatcatcttcttcttcatcccttccccaacttcatactcatccttcatcatcatcatcttcttcacattacataaaaaaatcacctttttttattaataaattcaataatcacaccaaatgcaaattccacaattcctctgctctgagaactaatttcttctacactaccataactaaaacctcatcatcaagattcgttctaaacaaaaacccaaaccaattttccgtcaaggcttgcagtcaagttggttctgctggatccgatccagcattgaataaagttgcagactttaaagatgcattttggaggtttctaaggccccatactattcgtgggacagcattaggatcagtgtctttagtaacgagagcactacttgaaaacccaaacttgattcggtggtcacttttgctcaaggcattttcaggtcttgttgctttgatatgtgggaatggttatatagtcgggatcaatcagatctatgatatcggtattgataaggtgaacaaaccatatttacctattgctgcgggagatctttctgtccagtcagcatggtttttggtgttagcatttgcaatggtaggcgttattattgttgggatgaacttcggcccattcatcacctccctttattctctcggtcttttcttgggcaccatctattccgttccaccacttcgaatgaagagatttcctgttgttgcatttcttatcatcgccacggtgagaggttttcttctaaattttggtgtgtattatgcggttagagcagctctgggactaacattccaatggagctcagcagtggcttttatcacaaccttcgttacattatttgctttagtcattgccattactaaagatcttcctgatgtagagggtgaccgaaagtttcaaatttctacttttgcaacaaaacttggagtaagaaacattgcattattagggtcaggacttctgctgatcaattatattgggtctatcgttgcagcactttacatgcctcaggtgaaaaccacttcgatagaccattacagaccatacagcttcctggttgatttaccaggtcaaaatgggattactttagcagcttga

SEQ ID NO: 6 - DNA, 1125 bp, Artificial Sequence
  Synthetic
atggctactatggcttcttctttgctgaatcctctttcttgttccattaaacccaactcaaacagactaccattaccattaccaatacccatttctctatctcgttcttgtagaaggctaacaatcaaagcaacggagacagatgcaaatgaagtgaagccaaaggcgccagagaaagcaccagctgcaagtggatctggttttaatcaaattcttgggattaaaggggctaaacaagaaactaataaatggaagatccgtgttcaacttacaaagccggttacttggcctccattaatttggggagtcgtatgtggagctgctgcttctggtaacttccaatggactgtggaagatgttgctaaatcaattgtttgcatgttgatgtctggcccatttctaaccggttacacacagacgatcaatgattggtatgatagagacattgatgctattaatgaaccttaccgtccaattccttccggagccatatctgaaaatgaggtcattactcaaatttgggtacttcttttaggaggcatcggattggctggtatattagacgtgtgggcagggcataagtcccctacaatattctatcttgctttgggtggatcattgttatcttatatctactcagctccacctttaaagctcaaacagaatggatggattggcaactttgcattaggagcaagctatattagcttaccatggtgggctggtcaagcattgttcggaactcttacacctgatatagtagttctcacacttttgtacagcatagctgggcttggtattgctatagtaaatgactttaaaagtgttgaaggagacaggaaaatggggcttcagtcccttcccgtggcttttggtgaagagacagctaaatggatatgtgttggtgccattgacataactcaactctctattgcaggttaccttttaggatctggtaaaccatattacgccttagcactcgttgggttgattgttccacaaatcttttttcagttcaagtactttcttaaagatccagttaaatatgatgtcaagtatcaggctagtgctcaaccatttctcattcttggtcttctggtgactgccttagctactagtcactga

SEQ ID NO: 7 - DNA, 1221 bp, Artificial Sequence
  Synthetic
atggcatctctagctattggttcacttggtagcccaagctcacgtcagtgttctagccccgttgcatcatcttcttcatttgcgatagggtcacaaatagcttcaaagtttcttcggatatcaaaatttgataagactaagaacagccccttaacattgcaacaaaagcatataaacaaaagcatagatcaaagcttctttgagccgcttccattgcacaaaataaacaaagacaagtttaagttgtatgcaacatctacaaacaatcctcagtttgatgcaactcatgatttgaagactccggaagtatccattatcaactttgtggacgctctttataggttaataaggccgtatacagcagttgtaacgatcgtaagtgtagtcgcgatgtcccttcttacagttaatagcctttcagatttttccccattgttcttcatcaaagtggtacaggctcttattggaggcatattcatgcaaatgtatgttagtggtttcaatcaaatttgtgatatagaactcgacaaggttaacaaacagtctcttccattagcggctggagaactatctatgaaaactgcgatcgtcatcgcatcactatcagctatcatgagcttatcgattggttggtttgttggctccccaccattattgtggtgtcttgtttggtggtttattgttgggactgcatattcggccaacgtgctgccttatttgcgatggaaaaggtttcctttcacagcagcattttgcgccatgacgtctcgggcactagttcttcctattggatattacttgcatatgcagaattccatcccgggagtatctgcattactttcaaggccaatattatttgcagtcgcaatgctcagtgcattttctttatcagcgatgttctttaaggacatccctgatattaagggagataggatgcatggaatcaagtctctagcaattaaactgggtgaaaaacgggtgtattggatttccatttcgattattgaaattgcttatattgctgctgcatttattggagcaacttcacccataagctggagcaagtatgtaacgattatcggtcatcttggaatgggattactactttgggtacgagccagatcagtagatccgacgaacacggtagccgttcaatcgatgtatatgttccttattaagctagtatatgcagaatacggacttatctcgcttgtacgctga

SEQ ID NO: 8 - DNA, 1188 bp, Artificial Sequence; identical to SEQ ID NO: 3
  Synthetic

SEQ ID NO: 9 - DNA, 1308 bp, Artificial Sequence
  Synthetic
atgttgattcaccatgaacattttttgacaaccggatttgaaagttcaaacgatcgagctgcttattcaataaacttttcgaaacaacatcacttacacatggcgtctatagctactggttcactttgtaggccaacctcacatcaattttctatccccgttgcatcatcttcttcatttgcgacaggatcacaattcgcttcaaagtttcttcatatatcaatatctgctaaaaaaagctcattgacattgcaacaaaggcatattcataaaaacatagatcaaagcttcttaaagccgcttgcacttcaaaaattgaacaaagacaagtttaagttgaatggaacatctccagacaatcctcagtttgatgcaactcatgatttgaagactcaaatagaatccactatcaactttgtggacgttctttataggttgttaaggccgtatgcattacttcaaatgggtttatgtgtagtcacgatgagtcttcttaccgttgaaagcctttcagatttttccccattgttcttcgtcaaagtggcacaggctcttattggaggcatattcatgcaaatgtatgttaatggttttaatcagatttgtgatatagaactcgacaaggttaacaaaccgtctcttccgttagcatctggggaactatctaagacaactactatagtcgtctcttcactatcagctattacgagcttatcgattggttggtttgttggctccccaccattgttgtggagtcttgttgtgtggtttattgctgggactacatattcggctaatctgccatatttgcgatggaaaaggtttcctttcacaaatatgttttgcaacttgacgatggcactagttgttcctattggaacttacttgcatatggagaattccatccacggagtatccacattactttcaaggccactattatttacagttgcaatgtgcactgtgtttcctgtttcgataatactctttaaggacatccctgatattaagggagaccggatgcatggaatgaagtctctagcaattatactgggtgaaaaacggacgtattggatatgcatttggattcttgaaatcacttatattgctgctgcttttttcggagcaacttcacccatcagctggagcaaatatgtaacgattattagtcatctaggaatggggttcttactttggctacgatccaaatcagtagatgtgaagaacacagtagccgttcaatctatgtatatgttcctttggaagctactctatgcagaatatggccttatcttgcttgtacgctga

SEQ ID NO: 10 - DNA, 1293 bp, Artificial Sequence
  Synthetic
atgtttattcaccatgaacagtttttgacaaccggatttgaaagttcaaacgatcgagctgcctattcaataaactttttgaaacaacatcacttacacatggtgtctatagctactggttcactttgtaggccaacctcacatcgattctctatccccgttgcatcatcttcttcatttgcgacaggatcacaattcgcttcaatatctgctaaaaaaagctcattgacattgaaacaaaggcatactcataaaaacatagatcaaagcttcttcaagccgcttgcacttcaaaaaatgaacaaaggcaagtttaagttgaatgcaacatctccagacaattctcagttggatgcaactcatgatttgaagactcaaatagaatccattatcaactttgtggacgttctttataggttgataaggccgtatgtagtacttggaatgggtgtaactatagtcacgatgtgtcttcttaccgttgatagcctttcagatttttccccattgttcttcgtcaaagtggcacaggctcttattggaagcatattcatggcaatgtatgttaatagttttaatgagatttgtgatatagaactcgacaaggttaacaaaccgtctcttccgttagcgtctggggaactatctatgacaactgctattgtcgtctcttcactatcagctatcatgagcttatcgattggttggtttgttggctccccaccattgttgtggagtcttgttgtgtggtttattcttgggactgcatattcggctaatctgccatatttgcgatggaaaaggtttcctttaacaacactgtcttccgccctgacgatgggggcactagttattcctattggaaattacatgcatatggagaattccatccgcggagtaaccacattactttcaaggccactattatttgcagttgcaatgtgcgctgcgtttcatgtttcgacgatactctttaaggacatccctgatattaagggagaccggatgcatggaatgaagtctctagcaattaaactgggtgaaaaacggatgtattggatatgcatttggattcttgaaatcgcttatattgctgctgcttttttcggagcaacttcacccatcagctggagcaaatatgtaacgattattagtcatctaggaatggggttcttactttggctacgatccaaatcagtagatgtgaagaacacagtagccgttcaatctatgtatatgttcctttggaagctattctatgtagaacatggtcttatcttgcttgtacgttga

SEQ ID NO: 11 - DNA, 1209 bp, Artificial Sequence
  Synthetic
atggcgtctatagctactggttcactttgtaggccaacctcacatcgattttctatccacgttgcatcatcttcttcatttgcgacaggatcacagtttgcttcaaagattcttcagatatcaatatctgctaaaaaaagctcattgacattgcaacaaaggcatattcataaaaacatagatcaaagcttcttcaagccgcttgcacttcaaaaaatgaacaaagacaagtttaagttgaatgcaacatctccagacaatccacagtttgatgcaactcgtgatttgaagactcaaatagaatccattatcaagtttgtggacgttctttataggttgttaaggccgtacgcaatacttgaaatgggtttaagtgtagtcacgatgagtcttcttaccgttgaaagcctttcagatttttccccgttgttcttcgtcaaagtggcacaagctcttattggaggcatattcatgcaaatgtatgttaatggttttaatcagatttgtgatatagaactcgacaaggttaacaaaccgtctcttccgttagcgtctggggaactatctacgacaactactatagtcgtctcttcactatcagctattatgagcttatcgattggttggtttgttggctccccaccattgttgtggagtcttgttgtgtggtttattgttgggacaacatattcgactaatctgccatatttgcgatggaaaaggtttcctttcacagcaatgttttgcaacctgacgagggcactagttgttcctattggaacttacttgcatatgaagaattccatccacgaagtatccacattactttcaaggccactgttatttgcagttgcaatgtgcactgtgtttcctatttcgataatactctttaaggacatccctgatattaagggagaccggatgcatggaatgaagtctctagcaattatactgggtgaagaacggacgtattggatatgcatttggattcttgaaatcgcttatattgctgctgcttttttcggagcaacttcacccatcagctggagcaaatatgtaatgattattagtcatctaggaatggggttcttactttggctacgatccaaatcagtagatgtgaagaacacagtagccgttcaatctatgtatatgttcctttggaagctactctatgcagaatatggccttattttgcttgtacgctga

SEQ ID NO: 12 - protein, 399 aa, Artificial Sequence
  Synthetic
MELSLSSSSSSSLPQLHTHPSSSSSSSHYIKKSPFFINKFNNHTKCKFHNSSALRTNFFYTTITKTSSSRFVLNKNPNQFSVKACSQVGSAGSDPALNKVADFKDAFWRFLRPHTIRGTALGSVSLVTRALLENPNLIRWSLLLKAFSGLVALICGNGYIVGINQIYDIGIDKVNKPYLPIAAGDLSVQSAWFLVLAFAMVGVIIVGMNFGPFITSLYSLGLFLGTIYSVPPLRMKRFPVVAFLIIATVRGFLLNFGVYYAVRAALGLTFQWSSAVAFITTFVTLFALVIAITKDLPDVEGDRKFQISTFATKLGVRNIALLGSGLLLINYIGSIVAALYMPQAFRSSLMIPLHTILASCLIYQAWILERANYTQEAIAGYYRFVWNLFYSEYIIFPFI

SEQ ID NO: 13 - protein, 372 aa, Artificial Sequence
  Synthetic
MATMASSLLNPLSCSIKPNSNRLPLPTPISLSRSCRRLTIKATETDANEVKPKAPEKAPAASGSGFNQILGIKGAKQETNKWKIRVQLTKPVTWPPLIWGVVCGAAASGNFQWTVEDVAKSIVCMLMSGPFLTGYTQTINDWYDRDIDAINEPYRPIPSGAISENEVITQIWVLLLGGIGLAGILDVWAGHKSPTIFYLALGGSLLSYIYSAPPLKLKQNGWIGNFALGASYISLPWWAGQALFGTLTPDIVVLTLLYSIAGLGIAIVNDFKSVEGDRKMGLQSLPVAFGEETAKWICVGAIDITQLSIAGYLLGSGKPYYALALVGLIVPQIFFQFKYFLKDPVKYDVKYQASAQPFLILGLLVTALATSH

SEQ ID NO: 14 - protein, 395 aa, Artificial Sequence
  Synthetic
MKSLIIGSFSNKVSCYSPSLPDSSSSLIPTGCYHVSLRTFQRNRAIQAQSSLVRCNIGKFNETLLLSRKRSTKHVACAVSEQPIEPDATNPQSSLPNALDAFYRFSRPHTVIGTALSIVSVSLLAVQKLSDFSPLFFIGVFEAIVAAFFMNIYIVGLNQLSDIEIDKVNKPYLPLASGEYSVQTGIIIVSSFAVMSFWLGWIVGSWPLFWALFISFLLGTAYSINIPMLRWKRFALVAAMCILAVRAIIVQVAFYLHIQTFVYGRLAVFPKPVIFATGFMSFFSVVIALFKDIPDIVGDKIFGIQSFTVRMGQKRVFWICILLLEIAYGVAILVGASSPFLWSRYITVLGHAILGLILWGRAKSTDLESKSAITSFYMFIWQLFYAEYLLIPLVR

SEQ ID NO: 15 - protein, 388 aa, Artificial Sequence
  Synthetic
MELSLSSSSSSSLPQLHTHPSSSSSSSHYIKKSPFFINKFNNHTKCKFHNSSALRTNFFYTTITKTSSSRFVLNKNPNQFSVKACSQVGSAGSDPALNKVADFKDAFWRFLRPHTIRGTALGSVSLVTRALLENPNLIRWSLLLKAFSGLVALICGNGYIVGINQIYDIGIDKVNKPYLPIAAGDLSVQSAWFLVLAFAMVGVIIVGMNFGPFITSLYSLGLFLGTIYSVPPLRMKRFPVVAFLIIATVRGFLLNFGVYYAVRAALGLTFQWSSAVAFITTFVTLFALVIAITKDLPDVEGDRKFQISTFATKLGVRNIALLGSGLLLINYIGSIVAALYMPQAFRSSLMIPLHTILASCLIYQAWILERANYTQRSQYFDMSSCRRR

SEQ ID NO: 16 - protein, 371 aa, Artificial Sequence
  Synthetic
MELSLSSSSSSSLPQLHTHPSSSSSSSHYIKKSPFFINKFNNHTKCKFHNSSALRTNFFYTTITKTSSSRFVLNKNPNQFSVKACSQVGSAGSDPALNKVADFKDAFWRFLRPHTIRGTALGSVSLVTRALLENPNLIRWSLLLKAFSGLVALICGNGYIVGINQIYDIGIDKVNKPYLPIAAGDLSVQSAWFLVLAFAMVGVIIVGMNFGPFITSLYSLGLFLGTIYSVPPLRMKRFPVVAFLIIATVRGFLLNFGVYYAVRAALGLTFQWSSAVAFITTFVTLFALVIAITKDLPDVEGDRKFQISTFATKLGVRNIALLGSGLLLINYIGSIVAALYMPQVKTTSIDHYRPYSFLVDLPGQNGITLAA

SEQ ID NO: 17 - protein, 374 aa, Artificial Sequence
  Synthetic
MATMASSLLNPLSCSIKPNSNRLPLPLPIPISLSRSCRRLTIKATETDANEVKPKAPEKAPAASGSGFNQILGIKGAKQETNKWKIRVQLTKPVTWPPLIWGVVCGAAASGNFQWTVEDVAKSIVCMLMSGPFLTGYTQTINDWYDRDIDAINEPYRPIPSGAISENEVITQIWVLLLGGIGLAGILDVWAGHKSPTIFYLALGGSLLSYIYSAPPLKLKQNGWIGNFALGASYISLPWWAGQALFGTLTPDIVVLTLLYSIAGLGIAIVNDFKSVEGDRKMGLQSLPVAFGEETAKWICVGAIDITQLSIAGYLLGSGKPYYALALVGLIVPQIFFQFKYFLKDPVKYDVKYQASAQPFLILGLLVTALATSH

SEQ ID NO: 18 - protein, 406 aa, Artificial Sequence
  Synthetic
MASLAIGSLGSPSSRQCSSPVASSSSFAIGSQIASKFLRISKFDKTKNSPLTLQQKHINKSIDQSFFEPLPLHKINKDKFKLYATSTNNPQFDATHDLKTPEVSIINFVDALYRLIRPYTAVVTIVSVVAMSLLTVNSLSDFSPLFFIKVVQALIGGIFMQMYVSGFNQICDIELDKVNKQSLPLAAGELSMKTAIVIASLSAIMSLSIGWFVGSPPLLWCLVWWFIVGTAYSANVLPYLRWKRFPFTAAFCAMTSRALVLPIGYYLHMQNSIPGVSALLSRPILFAVAMLSAFSLSAMFFKDIPDIKGDRMHGIKSLAIKLGEKRVYWISISIIEIAYIAAAFIGATSPISWSKYVTIIGHLGMGLLLWVRARSVDPTNTVAVQSMYMFLIKLVYAEYGLISLVR

SEQ ID NO: 19 - protein, 395 aa, Artificial Sequence; identical to SEQ ID NO: 14
  Synthetic

SEQ ID NO: 20 - protein, 435 aa, Artificial Sequence
  Synthetic
MLIHHEHFLTTGFESSNDRAAYSINFSKQHHLHMASIATGSLCRPTSHQFSIPVASSSSFATGSQFASKFLHISISAKKSSLTLQQRHIHKNIDQSFLKPLALQKLNKDKFKLNGTSPDNPQFDATHDLKTQIESTINFVDVLYRLLRPYALLQMGLCVVTMSLLTVESLSDFSPLFFVKVAQALIGGIFMQMYVNGFNQICDIELDKVNKPSLPLASGELSKTTTIVVSSLSAITSLSIGWFVGSPPLLWSLVVWFIAGTTYSANLPYLRWKRFPFTNMFCNLTMALVVPIGTYLHMENSIHGVSTLLSRPLLFTVAMCTVFPVSIILFKDIPDIKGDRMHGMKSLAIILGEKRTYWICIWILEITYIAAAFFGATSPISWSKYVTIISHLGMGFLLWLRSKSVDVKNTVAVQSMYMFLWKLLYAEYGLILLVR

SEQ ID NO: 21 - protein, 430 aa, Artificial Sequence
  Synthetic
MFIHHEQFLTTGFESSNDRAAYSINFLKQHHLHMVSIATGSLCRPTSHRFSIPVASSSSFATGSQFASISAKKSSLTLKQRHTHKNIDQSFFKPLALQKMNKGKFKLNATSPDNSQLDATHDLKTQIESIINFVDVLYRLIRPYVVLGMGVTIVTMCLLTVDSLSDFSPLFFVKVAQALIGSIFMAMYVNSFNEICDIELDKVNKPSLPLASGELSMTTAIVVSSLSAIMSLSIGWFVGSPPLLWSLVVWFILGTAYSANLPYLRWKRFPLTTLSSALTMGALVIPIGNYMHMENSIRGVTTLLSRPLLFAVAMCAAFHVSTILFKDIPDIKGDRMHGMKSLAIKLGEKRMYWICIWILEIAYIAAAFFGATSPISWSKYVTIISHLGMGFLLWLRSKSVDVKNTVAVQSMYMFLWKLFYVEHGLILLVR

SEQ ID NO: 22 - protein, 402 aa, Artificial Sequence
  Synthetic
MASIATGSLCRPTSHRFSIHVASSSSFATGSQFASKILQISISAKKSSLTLQQRHIHKNIDQSFFKPLALQKMNKDKFKLNATSPDNPQFDATRDLKTQIESIIKFVDVLYRLLRPYAILEMGLSVVTMSLLTVESLSDFSPLFFVKVAQALIGGIFMQMYVNGFNQICDIELDKVNKPSLPLASGELSTTTTIVVSSLSAIMSLSIGWFVGSPPLLWSLVVWFIVGTTYSTNLPYLRWKRFPFTAMFCNLTRALVVPIGTYLHMKNSIHEVSTLLSRPLLFAVAMCTVFPISIILFKDIPDIKGDRMHGMKSLAIILGEERTYWICIWILEIAYIAAAFFGATSPISWSKYVMIISHLGMGFLLWLRSKSVDVKNTVAVQSMYMFLWKLLYAEYGLILLVR

SEQ ID NO: 23 - DNA, 1221 bp, Artificial Sequence
  Synthetic
atggcatctctagctattggttcacttggtagcccaagctcacgtcagtgttctagccccgttgcatcatcttcttcatttgcgatagggtcacaaatagcttcaaagtttcttcggatatcaaaatttgataagactaagaacagccccttagcattgcaacaaaagcatataaacaaaagcatagatcaaagcttctttgagccgcttccattgcacaaaataaacaaagacaagtttaagttgtatgcaacatctacaaacaatcctcagtttgatgcaactcatgatttgaagactccggaagtatccattatcaactttgtggacgctctttataggttaataaggccgtatacagcagttgtaacgatcgtaagtgtagtcgcgatgtcccttcttacagttaatagcctttcagatttttccccattgttcttcatcaaagtggtacaggctcttattggaggcatattcatgcaaatgtatgttagtggtttcaatcaaatttgtgatatagaactcgacaaggttaacaaacagtctcttccattagcggctggagaactatctatgaaaactgcgatcgtcatcgcatcactatcagctatcatgagcttatcgattggttggtttgttggctccccaccattattgtggtgtcttgtttggtggtttattgttgggactgcatattcggccaacgtgctgccttatttgcgatggaaaaggtttcctttcacagcagcattttgcgccatgacgtctcgggcactagttcttcctattggatattacttgcatatgcagaattccatcccgggagtatctgcattactttcaaggccaatattatttgcagtcgcaatgctcagtgcattttctttatcagcgatgttctttaaggacatccctgatattaagggagataggatgcatggaatcaagtctctagcaattaaactgggtgaaaaacgggtgtattggatttccatttcgattattgaaattgcttatattgctgctgcatttattggagcaacttcacccataagctggagcaagtatgtaacgattatcggtcatcttggaatgggattactactttgggtacgagccagatcagtagatccgacgaacacggtagccgttcaatcgatgtatatgttccttattaagctagtatatgcagaatacggacttatctcgcttgtacgctga

SEQ ID NO: 24 - protein, 406 aa, Artificial Sequence
  Synthetic
MASLAIGSLGSPSSRQCSSPVASSSSFAIGSQIASKFLRISKFDKTKNSPLALQQKHINKSIDQSFFEPLPLHKINKDKFKLYATSTNNPQFDATHDLKTPEVSIINFVDALYRLIRPYTAVVTIVSVVAMSLLTVNSLSDFSPLFFIKVVQALIGGIFMQMYVSGFNQICDIELDKVNKQSLPLAAGELSMKTAIVIASLSAIMSLSIGWFVGSPPLLWCLVWWFIVGTAYSANVLPYLRWKRFPFTAAFCAMTSRALVLPIGYYLHMQNSIPGVSALLSRPILFAVAMLSAFSLSAMFFKDIPDIKGDRMHGIKSLAIKLGEKRVYWISISIIEIAYIAAAFIGATSPISWSKYVTIIGHLGMGLLLWVRARSVDPTNTVAVQSMYMFLIKLVYAEYGLISLVR

(19) United States
(12) Patent Application Publication
(10) Pub. No.: US 2024/0182873 A1
(43) Pub. Date: Jun. 6, 2024

(54) PRENYLTRANSFERASE AND A TRANSGENIC CELL, TISSUE, AND ORGANISM COMPRISING SAME

(71) Applicant: YEDA RESEARCH AND DEVELOPMENT CO. LTD., Rehovot (IL)

(72) Inventors: Asaph AHARONI, Tel Aviv (IL); Prashant SONAWANE, Rehovot (IL); Adam JOZWIAK, Rehovot (IL); Paula BERMAN, Rehovot (IL); Luis DE-HARO, Rehovot (IL)

(21) Appl. No.: 18/281,536

(22) PCT Filed: Mar. 10, 2022
(86) PCT No.: PCT/IL2022/050279

§ 371 Date: Sep. 11, 2023

Related U.S. Application Data
(60) Provisional application No. 63/159,028, filed on Mar. 10, 2021.

(51) Int. Cl. C12N 9/10; C12P 7/42

(52) U.S. Cl. CPC C12N 9/1085; C12P 7/42

(57) ABSTRACT

The present invention provides polynucleotide sequences derived from Helichrysum umbraculigerum and encoding a protein or a plurality thereof belonging to the prenyltransferase (PT) family. Further provided are an artificial nucleic acid molecule including the polynucleotide, a transgenic cell, tissue, or plant including same.

### CROSS-REFERENCE TO RELATED APPLICATIONS

This application claims the benefit of priority of U.S. Provisional Patent Application No. 63/159,028, titled “PRENYLTRANSFERASE AND A TRANSGENIC CELL, TISSUE, AND ORGANISM COMPRISING SAME”, filed Mar. 10, 2021, the contents of which are incorporated herein by reference in their entirety.

### FIELD OF INVENTION

The present invention relates to prenyl transferring enzymes (PT) including polynucleotides encoding same, and methods of using same.

### BACKGROUND

The development of novel methodologies related to natural products chemistry and biosynthesis is of growing interest. Prenylated aromatic natural products appear to be a very promising class of therapeutically active compounds. The prenylation of aromatic compounds often leads to significant alteration in the bioactivity profile of a compound, by both the creation of a novel C—C bond and also the introduction of one or more double bonds in the framework of the final product. Such compounds can affect a wide variety of biological systems in mammals and include roles as antioxidants, anti-inflammatories, anti-virals, anti-proliferatives, and anti-cancers.

Prenyltransferases are ubiquitous enzymes that catalyze the alkylation of electron rich prenyl acceptors by the alkyl moieties of allylic isoprene diphosphates. Prenyltransferases utilize isoprenoid diphosphates as substrates and catalyze the addition of the acyclic prenyl moiety to isopentenyl diphosphate (IPP), higher order prenyl diphosphates, aromatic rich molecules and proteins.

Prenyltransferases (PT) can also be useful in synthesis of cannabinoid analogs and synthesis of analogs of cannabinoid precursors. Cannabinoid analogs have been previously synthesized and may be useful as pharmaceutical products. There remains a need in the art to identify enzymes, and nucleotide sequences encoding such enzymes, e.g., PT, that are involved in the prenylation of aromatic acceptor molecules, e.g., aromatic polyketides.

There is a need in the art for the identification of novel enzymes capable of promoting the prenylation of aromatic compounds, as well as compounds which can modulate the prenylation of aromatic compounds. These and other needs are addressed by the present invention, as described in greater detail in the specification and claims which follow.

### SUMMARY

According to a first aspect, there is provided an isolated DNA molecule comprising a nucleic acid sequence having at least 91% homology to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or any combination thereof.

According to another aspect, there is provided an artificial nucleic acid molecule comprising the isolated DNA molecule of the invention.

According to another aspect, there is provided a plasmid or an agrobacterium comprising the artificial nucleic acid molecule disclosed herein.

According to another aspect, there is provided an isolated protein encoded by any one of: (a) the isolated DNA molecule of the invention; (b) the artificial vector disclosed herein; and (c) the plasmid or agrobacterium disclosed herein.

According to another aspect, there is provided a transgenic cell comprising: (a) the isolated DNA molecule of the invention; (b) the artificial nucleic acid molecule disclosed herein; (c) the plasmid or agrobacterium disclosed herein; (d) the isolated protein disclosed herein; or (e) any combination of (a) to (d).

According to another aspect, there is provided an extract derived from the transgenic cell disclosed herein, or any fraction thereof.

According to another aspect, there is provided a transgenic plant, a transgenic plant tissue or a plant part, comprising: (a) the isolated DNA molecule of the invention; (b) the artificial vector disclosed herein; (c) the plasmid or agrobacterium disclosed herein; (d) the isolated protein disclosed herein; (e) the transgenic cell disclosed herein; or (f) any combination of (a) to (e).

According to another aspect, there is provided a composition comprising: (a) the isolated DNA molecule of the invention; (b) the artificial vector disclosed herein; (c) the plasmid or agrobacterium disclosed herein; (d) the isolated protein disclosed herein; (e) the transgenic cell disclosed herein; (f) the extract disclosed herein; (g) the transgenic plant tissue or plant part disclosed herein; or (h) any combination of (a) to (g), and an acceptable carrier.

According to another aspect, there is provided a method for synthesizing a compound represented by Formula II:

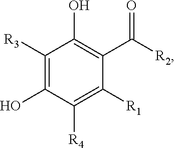

wherein: (i) R₁ is selected from the group consisting of: C1-C8 alkyl, and alpha-unsaturated phenylalkyl carboxylic acid, and R₂ is OH; or (ii) R₁ is OH and R₂ is selected from the group consisting of: C1-C8 alkyl, and alpha-unsaturated phenylalkyl carboxylic acid, wherein R₃ is a prenyl group, and wherein R₄ is hydrogen or a prenyl group, the method comprising: (a) providing a cell comprising an artificial vector comprising a nucleic acid sequence having at least 91% homology to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11; and (b) culturing the cell from step (a) such that a protein encoded by the artificial vector is expressed, thereby synthesizing the compound represented by Formula II.

According to another aspect, there is provided a method for synthesizing a compound represented by Formula II, the method comprising contacting a substrate molecule with an effective amount of a protein comprising an amino acid sequence with at least 92% homology to SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, or SEQ ID NO: 22, wherein the substrate molecule is represented by Formula I:

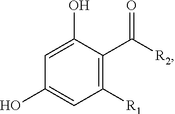

wherein: (i) R₁ is selected from the group consisting of: C1-C8 alkyl, and alpha-unsaturated phenylalkyl carboxylic acid; and R₂ is OH; or (ii) R₁ is OH and R₂ is selected from the group consisting of: C1-C8 alkyl, and alpha-unsaturated phenylalkyl carboxylic acid, thereby synthesizing the compound represented by Formula II.

According to another aspect, there is provided a method for obtaining an extract from a transgenic cell or a transfected cell comprising the steps: (a) culturing a transgenic cell or a transfected cell in a medium, wherein the transgenic cell or the transfected cell comprises an artificial vector comprising a nucleic acid sequence having at least 91% homology to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11; and (b) extracting the transgenic cell or the transfected cell, thereby obtaining an extract from the transgenic cell or the transfected cell.

According to another aspect, there is provided an extract of a transgenic cell or a transfected cell obtained according to the herein disclosed method.

According to another aspect, there is provided a medium or a portion thereof separated from a cultured transgenic cell or a cultured transfected cell, obtained according to the herein disclosed method.

According to another aspect, there is provided a composition comprising: (a) the extract disclosed herein; (b) the herein disclosed medium or a portion thereof; or (c) a combination of (a) and (b), and an acceptable carrier.

In some embodiments, the nucleic acid sequence has at least 80% homology to any one of SEQ ID Nos.: 1-11 is 950 to 1,750 nucleotides long.

In some embodiments, the nucleic acid sequence encodes a protein being a prenyl transferase.

In some embodiments, the transgenic cell is any one of: a unicellular organism, a cell of a multicellular organism, and a cell in a culture.

In some embodiments, the unicellular organism comprises a fungus or a bacterium.

In some embodiments, the fungus is a yeast cell.

In some embodiments, the extract comprises the isolated DNA molecule, the isolated protein, or both.

In some embodiments, the isolated protein comprises an amino acid sequence with at least 92% homology to SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, or SEQ ID NO: 22.

In some embodiments, the isolated protein consists of an amino acid sequence of SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, or SEQ ID NO: 22.

In some embodiments, the isolated protein is characterized by being capable of transferring a prenyl group to a substrate molecule.

In some embodiments, the prenyl group is selected from the group consisting of: dimethylallyl diphosphate, geranyl diphosphate, farnesyl diphosphate, and geranylgeranyl diphosphate.

In some embodiments, the substrate molecule is represented by Formula I.

In some embodiments, the alpha-unsaturated phenylalkyl carboxylic acid comprises cinnamic acid or a derivative thereof.

In some embodiments, the cinnamic acid derivative is a hydroxylated derivative of cinnamic acid.

In some embodiments, the hydroxylated derivative of cinnamic acid is coumaric acid.

In some embodiments, the transgenic plant is a Cannabis sativa plant.

In some embodiments, the protein is characterized by being capable of transferring a prenyl group to a substrate molecule.

In some embodiments, the culturing comprises supplementing the cell with an effective amount of the substrate molecule.

In some embodiments, the artificial vector is an expression vector.

In some embodiments, the cell is a prokaryote cell or a eukaryote cell.

In some embodiments, the cell is a transgenic cell, or a cell transfected with the isolated DNA molecule of the invention, or the artificial vector disclosed herein.

In some embodiments, the method further comprises a step preceding step (a), comprising introducing or transfecting the cell with the artificial vector.

In some embodiments, contacting is in a cell-free system.

In some embodiments, the substrate molecule is selected from the group consisting of: a resorcinoid precursor, a stilbene acid precursor, an acyl phloroglucinoid precursor, and a chalcone precursor.

In some embodiments, the substrate molecule is represented by a formula selected from the group consisting of:

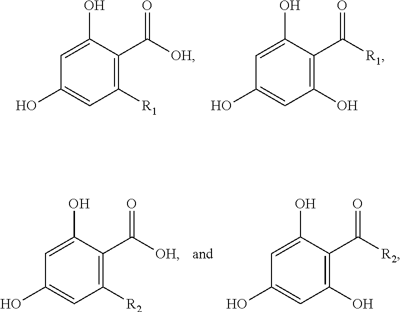

wherein R₁ is C1-C8 alkyl, and wherein R₂ is an alpha-unsaturated phenylalkyl carboxylic acid, or an alpha saturated phenylalkyl carboxylic acid.

In some embodiments, the substrate molecule is selected from the group consisting of:

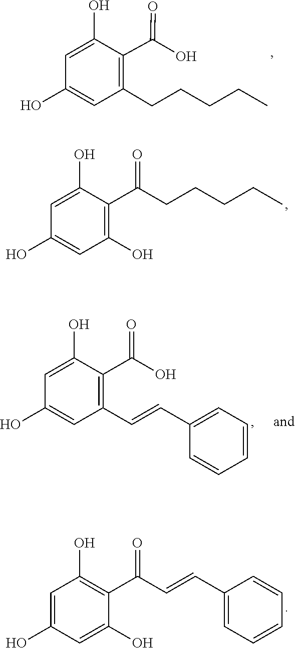 [SMILES: CCCCCc1cc(O)cc(O)c1C(=O)O ; CCCCCC(=O)c1c(O)cc(O)cc1O ; O=C(O)c1c(O)cc(O)cc1/C=C/c1ccccc1 ; O=C(/C=C/c1ccccc1)c1c(O)cc(O)cc1O]

In some embodiments, the compound is selected from the group consisting of: a cannabinoid, an amorfrutin, an acyl phlorogluconoid, and a prenyl chalcone.

In some embodiments, the compound is selected from the group consisting of:

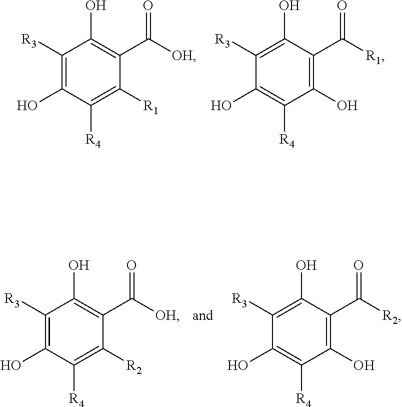

wherein: R₁ is C1-C8 alkyl, R₂ is an alpha-unsaturated phenylalkyl carboxylic acid or an alpha saturated phenylalkyl carboxylic acid, R₃ is a prenyl group, and R₄ is hydrogen or a prenyl group.

In some embodiments, the compound is selected from the group consisting of:

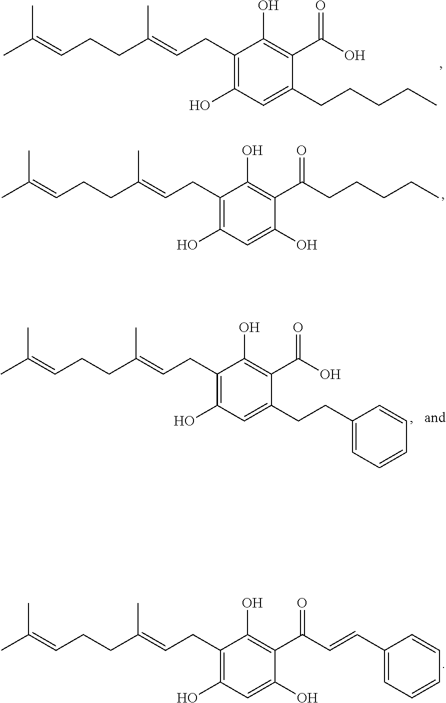

In some embodiments, the compound is

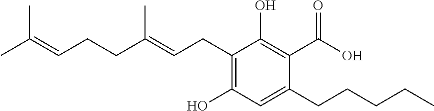 [SMILES: CCCCCc1cc(O)c(C/C=C(\C)CCC=C(C)C)c(O)c1C(=O)O]

In some embodiments, the method further comprises a step preceding step (b), comprising separating the cultured transgenic cell or the cultured transfected cell from the medium.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1B include graphs showing the identification of CBGA in a Helichrysum umbraculigerum ethanolic extract. (1A) Extracted ion current (XIC) chromatograms (359.222 Da), and (1B) MS/MS spectral matching of a CBGA standard versus a plant extract.

FIG. 2 includes a graph showing in vitro production of cannabigerolic acid (CBGA). Purified microsomal fraction from yeast cells expressing prenyltransferases (PTs) were used in enzyme assay containing olivetolic acid (OA) and geranylpyrophosphate (GPP). Cannabis sativa geranylpyrophosphate:olivetolate geranyltransferase 4 (CsGOT4) enzyme assay was used as positive control in experiment. Extracted ion chromatograms (EICs) are shown. LC-MS was used for assay products analysis. Standard—STD; Negative control—Empty vector.

FIG. 3 includes a phylogenetic tree of the PTs from Helichrysum umbraculigerum and functionally characterized aromatic PTs from other plants reviewed in de Brujin et al. (2020). Sequences were aligned using MUSCLE and a Maximum Likelihood tree using the JTT distance matrix-based method was constructed using MEGA11 software. Bootstrap values are indicated at the nodes of each branch (100 replicates). For comparison, GOT4 from Cannabis is presented (*).

FIG. 4 includes graphs showing steady state kinetic analysis of Helichrysum umbraculigerum PT1 (HuPT1; SEQ ID NO: 1), HuPT3 (SEQ ID NO: 3) and HuPTx (SEQ ID NO: 7) with olivetolic acid and GPP. The Michaelis-Menten Km value of each enzyme was calculated using varying olivetolic acid (0.5 μM-1.5 mM) and constant GPP (1 mM) concentrations (n=3 technically independent samples; measurements were plotted individually).

### DETAILED DESCRIPTION

The present invention, in some embodiments, is directed to polynucleotide sequences derived from Helichrysum umbraculigerum and encoding a protein or a plurality thereof belonging to the prenyltransferases (PT) family.

According to some embodiments, there is provided a polynucleotide comprising a nucleic acid sequence comprising any one of SEQ ID Nos.: 1-11, or any combination thereof.

In some embodiments, the polynucleotide is an isolated polynucleotide. In some embodiments, the polynucleotide is a DNA molecule. In some embodiments, the polynucleotide is an isolated DNA molecule. In some embodiments, the DNA molecule is an isolated DNA molecule. In some embodiments, the DNA molecule is a complementary DNA (cDNA) molecule.

As used herein, the terms “isolated polynucleotide” and “isolated DNA molecule” refers to a nucleic acid molecule that is essentially free from contaminating cellular components, such as carbohydrate, lipid, or other proteinaceous impurities associated with the nucleic acid in nature. Typically, a preparation of isolated DNA or RNA contains the nucleic acid in a highly purified form, e.g., at least about 80% pure, at least about 90% pure, at least about 95% pure, greater than 95% pure, or greater than 99% pure. In some embodiments, the isolated polynucleotide is any one of DNA, RNA, and cDNA. In some embodiments, the isolated polynucleotide is a synthesized polynucleotide. Synthesis of polynucleotides is well known in the art and may be performed, for example, by ligating or covalently linking by primer linkers multiple nucleic acid molecules together.

The term “nucleic acid” is well known in the art. A “nucleic acid” as used herein will generally refer to any molecule (e.g., a strand) of DNA, RNA or a derivative or analog thereof, comprising nucleotides. Nucleotides are comprised of nucleosides and phosphate groups. The nitrogenous bases of nucleosides include, for example, naturally occurring purine or pyrimidine nucleosides as found in DNA (e.g., an adenine “A,” a guanine “G,” a thymine “T” or a cytosine “C”) or RNA (e.g., an A, a G, an uracil “U” or a C).

The term “nucleic acid molecule” includes but is not limited to single-stranded RNA (ssRNA), double-stranded RNA (dsRNA), single-stranded DNA (ssDNA), double-stranded DNA (dsDNA), small RNAs, circular nucleic acids, fragments of genomic DNA or RNA, degraded nucleic acids, amplification products, modified nucleic acids, plasmid or organellar nucleic acids, and artificial nucleic acids such as oligonucleotides.

In some embodiments, the polynucleotide comprises or consists of the nucleic acid sequence:

(SEQ ID NO: 1)
ATGGAGTTATCACTCTCATCATCTTCTTCTTCATCCCTTCCCCAA
CTTCATACTCATCCTTCATCATCATCATCTTCTTCACATTACATA
AAAAAATCACCTTTTTTTATTAATAAATTCAATAATCACACCAAA
TGCAAATTCCACAATTCCTCTGCTCTGAGAACTAATTTCTTCTAC
ACTACCATAACTAAAACCTCATCATCAAGATTCGTTCTAAACAAA
AACCCAAACCAATTTTCCGTCAAGGCTTGCAGTCAAGTTGGTTCT
GCTGGATCCGATCCAGCATTGAATAAAGTTGCAGACTTTAAAGAT
GCATTTTGGAGGTTTCTAAGGCCCCATACTATTCGTGGGACAGCA
TTAGGATCAGTGTCTTTAGTAACGAGAGCACTACTTGAAAACCCA
AACTTGATTCGGTGGTCACTTTTGCTCAAGGCATTTTCAGGTCTT
GTTGCTTTGATATGTGGGAATGGTTATATAGTCGGGATCAATCAG
ATCTATGATATCGGTATTGATAAGGTGAACAAACCATATTTACCT
ATTGCTGCGGGAGATCTTTCTGTCCAGTCAGCATGGTTTTTGGTG
TTAGCATTTGCAATGGTAGGCGTTATTATTGTTGGGATGAACTTC
GGCCCATTCATCACCTCCCTTTATTCTCTCGGTCTTTTCTTGGGC
ACCATCTATTCCGTTCCACCACTTCGAATGAAGAGATTTCCTGTT
GTTGCATTTCTTATCATCGCCACGGTGAGAGGTTTTCTTCTAAAT
TTTGGTGTGTATTATGCGGTTAGAGCAGCTCTGGGACTAACATTC
CAATGGAGCTCAGCAGTGGCTTTTATCACAACCTTCGTTACATTA
TTTGCTTTAGTCATTGCCATTACTAAAGATCTTCCTGATGTAGAG
GGTGACCGAAAGTTTCAAATTTCTACTTTTGCAACAAAACTTGGA
GTAAGAAACATTGCATTATTAGGGTCAGGACTTCTGCTGATCAAT
TATATTGGGTCTATCGTTGCAGCACTTTACATGCCTCAGGCTTTC
AGGAGCAGCTTGATGATACCATTACATACCATATTAGCTTCCTGT
TTGATTTACCAGGCATGGATACTTGAGCGTGCGAATTACACCCAG
GAGGCGATAGCTGGGTACTACCGATTTGTATGGAATCTGTTTTAT
TCAGAGTACATCATATTTCCTTTCATCTGA.

In some embodiments, the polynucleotide comprises a nucleic acid sequence with at least 75%, at least 79%, at least 85%, at least 95%, or at least 99% homology or identity to SEQ ID NO: 1, or any value and range therebetween. Each possibility represents a separate embodiment of the invention. In some embodiments, the polynucleotide comprises a nucleic acid sequence with 75% to 100%, 80% to 100%, 90% to 100%, or 95% to 100% homology or identity to SEQ ID NO: 1. Each possibility represents a separate embodiment of the invention.

In some embodiments, the polynucleotide comprises or consists of the nucleic acid sequence:

(SEQ ID NO: 2)
ATGGCTACTATGGCTTCTTCTTTGCTGAATCCTCTTTCTTGTTCC
ATTAAACCCAACTCAAACAGACTACCATTACCAACACCCATTTCT
CTATCTCGTTCTTGTAGAAGGCTAACAATCAAAGCAACGGAGACA
GATGCAAATGAAGTGAAGCCAAAGGCGCCAGAGAAAGCACCAGCT
GCAAGTGGATCTGGTTTTAATCAAATTCTTGGGATTAAAGGGGCT
AAACAAGAAACTAATAAATGGAAGATCCGTGTTCAACTTACAAAG
CCGGTTACTTGGCCTCCATTAATTTGGGGAGTCGTATGTGGAGCT
GCTGCTTCTGGTAACTTCCAATGGACTGTGGAAGATGTTGCTAAA
TCAATTGTTTGCATGTTGATGTCTGGCCCATTTCTAACCGGTTAC
ACACAGACGATCAATGATTGGTATGATAGAGACATTGATGCTATT
AATGAACCTTACCGTCCAATTCCTTCCGGAGCCATATCTGAAAAT
GAGGTCATTACTCAAATTTGGGTACTTCTTTTAGGAGGCATCGGA
TTGGCTGGTATATTAGACGTGTGGGCAGGGCATAAGTCCCCTACA
ATATTCTATCTTGCTTTGGGTGGATCATTGTTATCTTATATCTAC
TCAGCTCCACCTTTAAAGCTCAAACAGAATGGATGGATTGGCAAC
TTTGCATTAGGAGCAAGCTATATTAGCTTACCATGGTGGGCTGGT
CAAGCATTGTTCGGAACTCTTACACCTGATATAGTAGTTCTCACA
CTTTTGTACAGCATAGCTGGGCTTGGTATTGCTATAGTAAATGAC
TTTAAAAGTGTTGAAGGAGACAGGAAAATGGGGCTTCAGTCCCTT
CCCGTGGCTTTTGGTGAAGAGACAGCTAAATGGATATGTGTTGGT
GCCATTGACATAACTCAACTCTCTATTGCAGGTTACCTTTTAGGA
TCTGGTAAACCATATTACGCCTTAGCACTCGTTGGGTTGATTGTT
CCACAAATCTTTTTTCAGTTCAAGTACTTTCTTAAAGATCCAGTT
AAATATGATGTCAAGTATCAGGCTAGTGCTCAACCATTTCTCATT
CTTGGTCTTCTGGTGACTGCCTTAGCTACTAGTCACTGA.

In some embodiments, the polynucleotide comprises a nucleic acid sequence with at least 80%, at least 85%, at least 90%, at least 95%, at least 97%, or at least 99% homology or identity to SEQ ID NO: 2, or any value and range therebetween. Each possibility represents a separate embodiment of the invention. In some embodiments, the polynucleotide comprises a nucleic acid sequence with 80% to 100%, 85% to 100%, 90% to 100%, or 95% to 100% homology or identity to SEQ ID NO: 2. Each possibility represents a separate embodiment of the invention.

In some embodiments, the polynucleotide comprises or consists of the nucleic acid sequence:

(SEQ ID NO: 3)
ATGAAGTCTTTGATTATTGGGTCTTTTTCTAATAAGGTTTCTTGT
TATTCCCCATCATTACCAGATTCATCTTCTTCACTTATACCAACA
GGTTGTTATCATGTATCACTAAGAACATTTCAGCGTAACCGAGCC
ATTCAAGCTCAATCAAGTCTTGTGAGATGCAATATTGGCAAATTC
AATGAAACATTACTACTTTCGCGGAAACGAAGTACAAAACATGTT
GCATGTGCGGTTTCTGAACAACCCATTGAACCAGATGCTACAAAC
CCTCAAAGTTCATTACCAAATGCTTTGGATGCTTTCTATAGGTTT
TCAAGACCTCATACAGTTATAGGAACTGCATTGAGCATAGTTTCG
GTTTCACTCCTAGCGGTTCAAAAGCTTTCGGATTTTTCTCCACTA
TTCTTCATTGGCGTTTTCGAGGCTATTGTTGCTGCCTTCTTTATG
AACATATACATTGTTGGCTTGAACCAGCTATCCGATATTGAAATA
GACAAGGTTAACAAGCCGTACCTTCCATTGGCATCTGGAGAATAT
TCAGTTCAAACTGGTATTATCATTGTATCATCATTTGCAGTCATG
AGTTTCTGGCTTGGATGGATCGTGGGCTCATGGCCTTTATTTTGG
GCACTTTTCATAAGTTTTCTTCTAGGGACCGCATATTCAATCAAT
ATACCGATGTTGAGATGGAAGCGCTTTGCTCTTGTGGCAGCAATG
TGTATTCTAGCTGTAAGAGCTATTATAGTTCAAGTTGCATTTTAT
TTGCACATTCAGACTTTTGTGTATGGAAGACTCGCCGTGTTCCCA
AAACCCGTGATATTTGCAACCGGATTTATGAGTTTCTTCTCTGTT
GTTATAGCATTGTTCAAGGACATACCCGACATTGTTGGAGACAAG
ATTTTTGGCATTCAATCATTTACTGTCCGTATGGGTCAAAAACGG
GTGTTTTGGATTTGCATCTTATTACTTGAAATAGCTTATGGTGTT
GCTATTCTAGTTGGGGCATCATCTCCCTTCCTTTGGAGCCGATAC
ATAACGGTATTGGGTCATGCGATTCTTGGTCTGATTCTCTGGGGT
CGTGCCAAGTCAACGGATCTGGAGAGCAAATCAGCAATAACCTCA
TTTTACATGTTCATATGGCAGTTGTTCTATGCCGAGTATTTGCTC
ATACCGCTCGTGAGATGA.

In some embodiments, the polynucleotide comprises a nucleic acid sequence with at least 75%, at least 79%, at least 85%, at least 95%, or at least 99% homology or identity to SEQ ID NO: 3, or any value and range therebetween. Each possibility represents a separate embodiment of the invention. In some embodiments, the polynucleotide comprises a nucleic acid sequence with 75% to 100%, 80% to 100%, 90% to 100%, or 95% to 100% homology or identity to SEQ ID NO: 3. Each possibility represents a separate embodiment of the invention.

In some embodiments, the polynucleotide comprises or consists of the nucleic acid sequence:

(SEQ ID NO: 4)
ATGGAGTTATCACTCTCATCATCTTCTTCTTCATCCCTTCCCCAA
CTTCATACTCATCCTTCATCATCATCATCTTCTTCACATTACATA
AAAAAATCACCTTTTTTTATTAATAAATTCAATAATCACACCAAA
TGCAAATTCCACAATTCCTCTGCTCTGAGAACTAATTTCTTCTAC
ACTACCATAACTAAAACCTCATCATCAAGATTCGTTCTAAACAAA
AACCCAAACCAATTTTCCGTCAAGGCTTGCAGTCAAGTTGGTTCT
GCTGGATCCGATCCAGCATTGAATAAAGTTGCAGACTTTAAAGAT
GCATTTTGGAGGTTTCTAAGGCCCCATACTATTCGTGGGACAGCA
TTAGGATCAGTGTCTTTAGTAACGAGAGCACTACTTGAAAACCCA
AACTTGATTCGGTGGTCACTTTTGCTCAAGGCATTTTCAGGTCTT
GTTGCTTTGATATGTGGGAATGGTTATATAGTCGGGATCAATCAG
ATCTATGATATCGGTATTGATAAGGTGAACAAACCATATTTACCT
ATTGCTGCGGGAGATCTTTCTGTCCAGTCAGCATGGTTTTTGGTG
TTAGCATTTGCAATGGTAGGCGTTATTATTGTTGGGATGAACTTC
GGCCCATTCATCACCTCCCTTTATTCTCTCGGTCTTTTCTTGGGC
ACCATCTATTCCGTTCCACCACTTCGAATGAAGAGATTTCCTGTT
GTTGCATTTCTTATCATCGCCACGGTGAGAGGTTTTCTTCTAAAT
TTTGGTGTGTATTATGCGGTTAGAGCAGCTCTGGGACTAACATTC
CAATGGAGCTCAGCAGTGGCTTTTATCACAACCTTCGTTACATTA
TTTGCTTTAGTCATTGCCATTACTAAAGATCTTCCTGATGTAGAG
GGTGACCGAAAGTTTCAAATTTCTACTTTTGCAACAAAACTTGGA
GTAAGAAACATTGCATTATTAGGGTCAGGACTTCTGCTGATCAAT
TATATTGGGTCTATCGTTGCAGCACTTTACATGCCTCAGGCTTTC
AGGAGCAGCTTGATGATACCATTACATACCATATTAGCTTCCTGT
TTGATTTACCAGGCATGGATACTTGAGCGTGCGAATTACACCCAG
CGATCACAGTACTTTGACATGTCATCTTGCAGGAGGCGATAG.

In some embodiments, the polynucleotide comprises a nucleic acid sequence with at least 91%, at least 92%, at least 95%, at least 97%, or at least 99% homology or identity to SEQ ID NO: 4, or any value and range therebetween. Each possibility represents a separate embodiment of the invention. In some embodiments, the polynucleotide comprises a nucleic acid sequence with 91% to 100%, 93% to 100%, 95% to 100%, or 97% to 100% homology or identity to SEQ ID NO: 4. Each possibility represents a separate embodiment of the invention.

In some embodiments, the polynucleotide comprises or consists of the nucleic acid sequence:

(SEQ ID NO: 5)
ATGGAGTTATCACTCTCATCATCTTCTTCTTCATCCCTTCCCCAA
CTTCATACTCATCCTTCATCATCATCATCTTCTTCACATTACATA
AAAAAATCACCTTTTTTTATTAATAAATTCAATAATCACACCAAA
TGCAAATTCCACAATTCCTCTGCTCTGAGAACTAATTTCTTCTAC
ACTACCATAACTAAAACCTCATCATCAAGATTCGTTCTAAACAAA
AACCCAAACCAATTTTCCGTCAAGGCTTGCAGTCAAGTTGGTTCT
GCTGGATCCGATCCAGCATTGAATAAAGTTGCAGACTTTAAAGAT
GCATTTTGGAGGTTTCTAAGGCCCCATACTATTCGTGGGACAGCA
TTAGGATCAGTGTCTTTAGTAACGAGAGCACTACTTGAAAACCCA
AACTTGATTCGGTGGTCACTTTTGCTCAAGGCATTTTCAGGTCTT
GTTGCTTTGATATGTGGGAATGGTTATATAGTCGGGATCAATCAG
ATCTATGATATCGGTATTGATAAGGTGAACAAACCATATTTACCT
ATTGCTGCGGGAGATCTTTCTGTCCAGTCAGCATGGTTTTTGGTG
TTAGCATTTGCAATGGTAGGCGTTATTATTGTTGGGATGAACTTC
GGCCCATTCATCACCTCCCTTTATTCTCTCGGTCTTTTCTTGGGC
ACCATCTATTCCGTTCCACCACTTCGAATGAAGAGATTTCCTGTT
GTTGCATTTCTTATCATCGCCACGGTGAGAGGTTTTCTTCTAAAT
TTTGGTGTGTATTATGCGGTTAGAGCAGCTCTGGGACTAACATTC
CAATGGAGCTCAGCAGTGGCTTTTATCACAACCTTCGTTACATTA
TTTGCTTTAGTCATTGCCATTACTAAAGATCTTCCTGATGTAGAG
GGTGACCGAAAGTTTCAAATTTCTACTTTTGCAACAAAACTTGGA
GTAAGAAACATTGCATTATTAGGGTCAGGACTTCTGCTGATCAAT
TATATTGGGTCTATCGTTGCAGCACTTTACATGCCTCAGGTGAAA
ACCACTTCGATAGACCATTACAGACCATACAGCTTCCTGGTTGAT
TTACCAGGTCAAAATGGGATTACTTTAGCAGCTTGA.

In some embodiments, the polynucleotide comprises a nucleic acid sequence with at least 91%, at least 92%, at least 95%, at least 97%, or at least 99% homology or identity to SEQ ID NO: 5, or any value and range therebetween. Each possibility represents a separate embodiment of the invention. In some embodiments, the polynucleotide comprises a nucleic acid sequence with 91% to 100%, 93% to 100%, 95% to 100%, or 97% to 100% homology or identity to SEQ ID NO: 5. Each possibility represents a separate embodiment of the invention.

In some embodiments, the polynucleotide comprises or consists of the nucleic acid sequence:

(SEQ ID NO: 6)
ATGGCTACTATGGCTTCTTCTTTGCTGAATCCTCTTTCTTGTTCC
ATTAAACCCAACTCAAACAGACTACCATTACCATTACCAATACCC
ATTTCTCTATCTCGTTCTTGTAGAAGGCTAACAATCAAAGCAACG
GAGACAGATGCAAATGAAGTGAAGCCAAAGGCGCCAGAGAAAGCA
CCAGCTGCAAGTGGATCTGGTTTTAATCAAATTCTTGGGATTAAA
GGGGCTAAACAAGAAACTAATAAATGGAAGATCCGTGTTCAACTT
ACAAAGCCGGTTACTTGGCCTCCATTAATTTGGGGAGTCGTATGT
GGAGCTGCTGCTTCTGGTAACTTCCAATGGACTGTGGAAGATGTT
GCTAAATCAATTGTTTGCATGTTGATGTCTGGCCCATTTCTAACC
GGTTACACACAGACGATCAATGATTGGTATGATAGAGACATTGAT
GCTATTAATGAACCTTACCGTCCAATTCCTTCCGGAGCCATATCT
GAAAATGAGGTCATTACTCAAATTTGGGTACTTCTTTTAGGAGGC
ATCGGATTGGCTGGTATATTAGACGTGTGGGCAGGGCATAAGTCC
CCTACAATATTCTATCTTGCTTTGGGTGGATCATTGTTATCTTAT
ATCTACTCAGCTCCACCTTTAAAGCTCAAACAGAATGGATGGATT
GGCAACTTTGCATTAGGAGCAAGCTATATTAGCTTACCATGGTGG
GCTGGTCAAGCATTGTTCGGAACTCTTACACCTGATATAGTAGTT
CTCACACTTTTGTACAGCATAGCTGGGCTTGGTATTGCTATAGTA
AATGACTTTAAAAGTGTTGAAGGAGACAGGAAAATGGGGCTTCAG
TCCCTTCCCGTGGCTTTTGGTGAAGAGACAGCTAAATGGATATGT
GTTGGTGCCATTGACATAACTCAACTCTCTATTGCAGGTTACCTT
TTAGGATCTGGTAAACCATATTACGCCTTAGCACTCGTTGGGTTG
ATTGTTCCACAAATCTTTTTTCAGTTCAAGTACTTTCTTAAAGAT
CCAGTTAAATATGATGTCAAGTATCAGGCTAGTGCTCAACCATTT
CTCATTCTTGGTCTTCTGGTGACTGCCTTAGCTACTAGTCACTGA.

In some embodiments, the polynucleotide comprises a nucleic acid sequence with at least 90%, at least 92%, at least 95%, at least 97%, or at least 99% homology or identity to SEQ ID NO: 6, or any value and range therebetween. Each possibility represents a separate embodiment of the invention. In some embodiments, the polynucleotide comprises a nucleic acid sequence with 90% to 100%, 92% to 100%, 95% to 100%, or 97% to 100% homology or identity to SEQ ID NO: 6. Each possibility represents a separate embodiment of the invention.

In some embodiments, the polynucleotide comprises or consists of the nucleic acid sequence:

(SEQ ID NO: 7)
ATGGCATCTCTAGCTATTGGTTCACTTGGTAGCCCAAGCTCACGT
CAGTGTTCTAGCCCCGTTGCATCATCTTCTTCATTTGCGATAGGG
TCACAAATAGCTTCAAAGTTTCTTCGGATATCAAAATTTGATAAG
ACTAAGAACAGCCCCTTAACATTGCAACAAAAGCATATAAACAAA
AGCATAGATCAAAGCTTCTTTGAGCCGCTTCCATTGCACAAAATA
AACAAAGACAAGTTTAAGTTGTATGCAACATCTACAAACAATCCT
CAGTTTGATGCAACTCATGATTTGAAGACTCCGGAAGTATCCATT
ATCAACTTTGTGGACGCTCTTTATAGGTTAATAAGGCCGTATACA
GCAGTTGTAACGATCGTAAGTGTAGTCGCGATGTCCCTTCTTACA
GTTAATAGCCTTTCAGATTTTTCCCCATTGTTCTTCATCAAAGTG
GTACAGGCTCTTATTGGAGGCATATTCATGCAAATGTATGTTAGT
GGTTTCAATCAAATTTGTGATATAGAACTCGACAAGGTTAACAAA
CAGTCTCTTCCATTAGCGGCTGGAGAACTATCTATGAAAACTGCG
ATCGTCATCGCATCACTATCAGCTATCATGAGCTTATCGATTGGT
TGGTTTGTTGGCTCCCCACCATTATTGTGGTGTCTTGTTTGGTGG
TTTATTGTTGGGACTGCATATTCGGCCAACGTGCTGCCTTATTTG
CGATGGAAAAGGTTTCCTTTCACAGCAGCATTTTGCGCCATGACG
TCTCGGGCACTAGTTCTTCCTATTGGATATTACTTGCATATGCAG
AATTCCATCCCGGGAGTATCTGCATTACTTTCAAGGCCAATATTA
TTTGCAGTCGCAATGCTCAGTGCATTTTCTTTATCAGCGATGTTC
TTTAAGGACATCCCTGATATTAAGGGAGATAGGATGCATGGAATC
AAGTCTCTAGCAATTAAACTGGGTGAAAAACGGGTGTATTGGATT
TCCATTTCGATTATTGAAATTGCTTATATTGCTGCTGCATTTATT
GGAGCAACTTCACCCATAAGCTGGAGCAAGTATGTAACGATTATC
GGTCATCTTGGAATGGGATTACTACTTTGGGTACGAGCCAGATCA
GTAGATCCGACGAACACGGTAGCCGTTCAATCGATGTATATGTTC
CTTATTAAGCTAGTATATGCAGAATACGGACTTATCTCGCTTGTA
CGCTGA.

In some embodiments, the polynucleotide comprises a nucleic acid sequence with at least 77%, at least 79%, at least 85%, at least 95%, or at least 99% homology or identity to SEQ ID NO: 7, or any value and range therebetween. Each possibility represents a separate embodiment of the invention. In some embodiments, the polynucleotide comprises a nucleic acid sequence with 77% to 100%, 85% to 100%, 90% to 100%, or 95% to 100% homology or identity to SEQ ID NO: 7. Each possibility represents a separate embodiment of the invention.

In some embodiments, the polynucleotide comprises or consists of the nucleic acid sequence:

(SEQ ID NO: 8)
ATGAAGTCTTTGATTATTGGGTCTTTTTCTAATAAGGTTTCTTGT
TATTCCCCATCATTACCAGATTCATCTTCTTCACTTATACCAACA
GGTTGTTATCATGTATCACTAAGAACATTTCAGCGTAACCGAGCC
ATTCAAGCTCAATCAAGTCTTGTGAGATGCAATATTGGCAAATTC
AATGAAACATTACTACTTTCGCGGAAACGAAGTACAAAACATGTT
GCATGTGCGGTTTCTGAACAACCCATTGAACCAGATGCTACAAAC
CCTCAAAGTTCATTACCAAATGCTTTGGATGCTTTCTATAGGTTT
TCAAGACCTCATACAGTTATAGGAACTGCATTGAGCATAGTTTCG
GTTTCACTCCTAGCGGTTCAAAAGCTTTCGGATTTTTCTCCACTA
TTCTTCATTGGCGTTTTCGAGGCTATTGTTGCTGCCTTCTTTATG
AACATATACATTGTTGGCTTGAACCAGCTATCCGATATTGAAATA
GACAAGGTTAACAAGCCGTACCTTCCATTGGCATCTGGAGAATAT
TCAGTTCAAACTGGTATTATCATTGTATCATCATTTGCAGTCATG
AGTTTCTGGCTTGGATGGATCGTGGGCTCATGGCCTTTATTTTGG
GCACTTTTCATAAGTTTTCTTCTAGGGACCGCATATTCAATCAAT
ATACCGATGTTGAGATGGAAGCGCTTTGCTCTTGTGGCAGCAATG
TGTATTCTAGCTGTAAGAGCTATTATAGTTCAAGTTGCATTTTAT
TTGCACATTCAGACTTTTGTGTATGGAAGACTCGCCGTGTTCCCA
AAACCCGTGATATTTGCAACCGGATTTATGAGTTTCTTCTCTGTT
GTTATAGCATTGTTCAAGGACATACCCGACATTGTTGGAGACAAG
ATTTTTGGCATTCAATCATTTACTGTCCGTATGGGTCAAAAACGG
GTGTTTTGGATTTGCATCTTATTACTTGAAATAGCTTATGGTGTT
GCTATTCTAGTTGGGGCATCATCTCCCTTCCTTTGGAGCCGATAC
ATAACGGTATTGGGTCATGCGATTCTTGGTCTGATTCTCTGGGGT
CGTGCCAAGTCAACGGATCTGGAGAGCAAATCAGCAATAACCTCA
TTTTACATGTTCATATGGCAGTTGTTCTATGCCGAGTATTTGCTC
ATACCGCTCGTGAGATGA.

In some embodiments, the polynucleotide comprises a nucleic acid sequence with at least 89%, at least 90%, at least 92%, at least 95%, at least 96%, at least 97%, at least 98%, or at least 99% homology or identity to SEQ ID NO: 8, or any value and range therebetween. Each possibility represents a separate embodiment of the invention. In some embodiments, the polynucleotide comprises a nucleic acid sequence with 89% to 100%, 92% to 100%, 94% to 100%, or 97% to 100% homology or identity to SEQ ID NO: 8. Each possibility represents a separate embodiment of the invention.

In some embodiments, the polynucleotide comprises or consists of the nucleic acid sequence:

(SEQ ID NO: 9)
ATGTTGATTCACCATGAACATTTTTTGACAACCGGATTTGAAAGT
TCAAACGATCGAGCTGCTTATTCAATAAACTTTTCGAAACAACAT
CACTTACACATGGCGTCTATAGCTACTGGTTCACTTTGTAGGCCA
ACCTCACATCAATTTTCTATCCCCGTTGCATCATCTTCTTCATTT
GCGACAGGATCACAATTCGCTTCAAAGTTTCTTCATATATCAATA
TCTGCTAAAAAAAGCTCATTGACATTGCAACAAAGGCATATTCAT
AAAAACATAGATCAAAGCTTCTTAAAGCCGCTTGCACTTCAAAAA
TTGAACAAAGACAAGTTTAAGTTGAATGGAACATCTCCAGACAAT
CCTCAGTTTGATGCAACTCATGATTTGAAGACTCAAATAGAATCC
ACTATCAACTTTGTGGACGTTCTTTATAGGTTGTTAAGGCCGTAT
GCATTACTTCAAATGGGTTTATGTGTAGTCACGATGAGTCTTCTT
ACCGTTGAAAGCCTTTCAGATTTTTCCCCATTGTTCTTCGTCAAA
GTGGCACAGGCTCTTATTGGAGGCATATTCATGCAAATGTATGTT
AATGGTTTTAATCAGATTTGTGATATAGAACTCGACAAGGTTAAC
AAACCGTCTCTTCCGTTAGCATCTGGGGAACTATCTAAGACAACT
ACTATAGTCGTCTCTTCACTATCAGCTATTACGAGCTTATCGATT
GGTTGGTTTGTTGGCTCCCCACCATTGTTGTGGAGTCTTGTTGTG
TGGTTTATTGCTGGGACTACATATTCGGCTAATCTGCCATATTTG
CGATGGAAAAGGTTTCCTTTCACAAATATGTTTTGCAACTTGACG
ATGGCACTAGTTGTTCCTATTGGAACTTACTTGCATATGGAGAAT
TCCATCCACGGAGTATCCACATTACTTTCAAGGCCACTATTATTT
ACAGTTGCAATGTGCACTGTGTTTCCTGTTTCGATAATACTCTTT
AAGGACATCCCTGATATTAAGGGAGACCGGATGCATGGAATGAAG
TCTCTAGCAATTATACTGGGTGAAAAACGGACGTATTGGATATGC
ATTTGGATTCTTGAAATCACTTATATTGCTGCTGCTTTTTTCGGA
GCAACTTCACCCATCAGCTGGAGCAAATATGTAACGATTATTAGT
CATCTAGGAATGGGGTTCTTACTTTGGCTACGATCCAAATCAGTA
GATGTGAAGAACACAGTAGCCGTTCAATCTATGTATATGTTCCTT
TGGAAGCTACTCTATGCAGAATATGGCCTTATCTTGCTTGTACGC
TGA.

In some embodiments, the polynucleotide comprises a nucleic acid sequence with at least 76%, at least 80%, at least 85%, at least 90%, at least 95%, at least 97%, at least 98%, or at least 99% homology or identity to SEQ ID NO: 9, or any value and range therebetween. Each possibility represents a separate embodiment of the invention. In some embodiments, the polynucleotide comprises a nucleic acid sequence with 76% to 100%, 83% to 100%, 90% to 100%, or 95% to 100% homology or identity to SEQ ID NO: 9. Each possibility represents a separate embodiment of the invention.

In some embodiments, the polynucleotide comprises or consists of the nucleic acid sequence:

(SEQ ID NO: 10)
ATGTTTATTCACCATGAACAGTTTTTGACAACCGGATTTGAAAGTTCAAA
CGATCGAGCTGCCTATTCAATAAACTTTTTGAAACAACATCACTTACACA
TGGTGTCTATAGCTACTGGTTCACTTTGTAGGCCAACCTCACATCGATTC
TCTATCCCCGTTGCATCATCTTCTTCATTTGCGACAGGATCACAATTCGC
TTCAATATCTGCTAAAAAAAGCTCATTGACATTGAAACAAAGGCATACTC
ATAAAAACATAGATCAAAGCTTCTTCAAGCCGCTTGCACTTCAAAAAATG
AACAAAGGCAAGTTTAAGTTGAATGCAACATCTCCAGACAATTCTCAGTT
GGATGCAACTCATGATTTGAAGACTCAAATAGAATCCATTATCAACTTTG
TGGACGTTCTTTATAGGTTGATAAGGCCGTATGTAGTACTTGGAATGGGT
GTAACTATAGTCACGATGTGTCTTCTTACCGTTGATAGCCTTTCAGATTT
TTCCCCATTGTTCTTCGTCAAAGTGGCACAGGCTCTTATTGGAAGCATAT
TCATGGCAATGTATGTTAATAGTTTTAATGAGATTTGTGATATAGAACTC
GACAAGGTTAACAAACCGTCTCTTCCGTTAGCGTCTGGGGAACTATCTAT
GACAACTGCTATTGTCGTCTCTTCACTATCAGCTATCATGAGCTTATCGA
TTGGTTGGTTTGTTGGCTCCCCACCATTGTTGTGGAGTCTTGTTGTGTGG
TTTATTCTTGGGACTGCATATTCGGCTAATCTGCCATATTTGCGATGGAA
AAGGTTTCCTTTAACAACACTGTCTTCCGCCCTGACGATGGGGGCACTAG
TTATTCCTATTGGAAATTACATGCATATGGAGAATTCCATCCGCGGAGTA
ACCACATTACTTTCAAGGCCACTATTATTTGCAGTTGCAATGTGCGCTGC
GTTTCATGTTTCGACGATACTCTTTAAGGACATCCCTGATATTAAGGGAG
ACCGGATGCATGGAATGAAGTCTCTAGCAATTAAACTGGGTGAAAAACGG
ATGTATTGGATATGCATTTGGATTCTTGAAATCGCTTATATTGCTGCTGC
TTTTTTCGGAGCAACTTCACCCATCAGCTGGAGCAAATATGTAACGATTA
TTAGTCATCTAGGAATGGGGTTCTTACTTTGGCTACGATCCAAATCAGTA
GATGTGAAGAACACAGTAGCCGTTCAATCTATGTATATGTTCCTTTGGAA
GCTATTCTATGTAGAACATGGTCTTATCTTGCTTGTACGTTGA.

In some embodiments, the polynucleotide comprises a nucleic acid sequence with at least 75%, at least 80%, at least 85%, at least 90%, at least 95%, at least 97%, at least 98%, or at least 99% homology or identity to SEQ ID NO: 10, or any value and range therebetween. Each possibility represents a separate embodiment of the invention. In some embodiments, the polynucleotide comprises a nucleic acid sequence with 75% to 100%, 80% to 100%, 90% to 100%, or 95% to 100% homology or identity to SEQ ID NO: 10. Each possibility represents a separate embodiment of the invention.

In some embodiments, the polynucleotide comprises or consists of the nucleic acid sequence:

(SEQ ID NO: 11)
ATGGCGTCTATAGCTACTGGTTCACTTTGTAGGCCAACCTCACATCGATT
TTCTATCCACGTTGCATCATCTTCTTCATTTGCGACAGGATCACAGTTTG
CTTCAAAGATTCTTCAGATATCAATATCTGCTAAAAAAAGCTCATTGACA
TTGCAACAAAGGCATATTCATAAAAACATAGATCAAAGCTTCTTCAAGCC
GCTTGCACTTCAAAAAATGAACAAAGACAAGTTTAAGTTGAATGCAACAT
CTCCAGACAATCCACAGTTTGATGCAACTCGTGATTTGAAGACTCAAATA
GAATCCATTATCAAGTTTGTGGACGTTCTTTATAGGTTGTTAAGGCCGTA
CGCAATACTTGAAATGGGTTTAAGTGTAGTCACGATGAGTCTTCTTACCG
TTGAAAGCCTTTCAGATTTTTCCCCGTTGTTCTTCGTCAAAGTGGCACAA
GCTCTTATTGGAGGCATATTCATGCAAATGTATGTTAATGGTTTTAATCA
GATTTGTGATATAGAACTCGACAAGGTTAACAAACCGTCTCTTCCGTTAG
CGTCTGGGGAACTATCTACGACAACTACTATAGTCGTCTCTTCACTATCA
GCTATTATGAGCTTATCGATTGGTTGGTTTGTTGGCTCCCCACCATTGTT
GTGGAGTCTTGTTGTGTGGTTTATTGTTGGGACAACATATTCGACTAATC
TGCCATATTTGCGATGGAAAAGGTTTCCTTTCACAGCAATGTTTTGCAAC
CTGACGAGGGCACTAGTTGTTCCTATTGGAACTTACTTGCATATGAAGAA
TTCCATCCACGAAGTATCCACATTACTTTCAAGGCCACTGTTATTTGCAG
TTGCAATGTGCACTGTGTTTCCTATTTCGATAATACTCTTTAAGGACATC
CCTGATATTAAGGGAGACCGGATGCATGGAATGAAGTCTCTAGCAATTAT
ACTGGGTGAAGAACGGACGTATTGGATATGCATTTGGATTCTTGAAATCG
CTTATATTGCTGCTGCTTTTTTCGGAGCAACTTCACCCATCAGCTGGAGC
AAATATGTAATGATTATTAGTCATCTAGGAATGGGGTTCTTACTTTGGCT
ACGATCCAAATCAGTAGATGTGAAGAACACAGTAGCCGTTCAATCTATGT
ATATGTTCCTTTGGAAGCTACTCTATGCAGAATATGGCCTTATTTTGCTT
GTACGCTGA.

In some embodiments, the polynucleotide comprises a nucleic acid sequence with at least 76%, at least 80%, at least 85%, at least 90%, at least 95%, at least 97%, at least 98%, or at least 99% homology or identity to SEQ ID NO: 11, or any value and range therebetween. Each possibility represents a separate embodiment of the invention. In some embodiments, the polynucleotide comprises a nucleic acid sequence with 76% to 100%, 85% to 100%, 90% to 100%, or 96% to 100% homology or identity to SEQ ID NO: 11. Each possibility represents a separate embodiment of the invention.

In some embodiments, the polynucleotide comprises or consists of the nucleic acid sequence:

(SEQ ID NO: 23)
ATGGCATCTCTAGCTATTGGTTCACTTGGTAGCCCAAGCTCACGTCAGTG
TTCTAGCCCCGTTGCATCATCTTCTTCATTTGCGATAGGGTCACAAATAG
CTTCAAAGTTTCTTCGGATATCAAAATTTGATAAGACTAAGAACAGCCCC
TTAGCATTGCAACAAAAGCATATAAACAAAAGCATAGATCAAAGCTTCTT
TGAGCCGCTTCCATTGCACAAAATAAACAAAGACAAGTTTAAGTTGTATG
CAACATCTACAAACAATCCTCAGTTTGATGCAACTCATGATTTGAAGACT
CCGGAAGTATCCATTATCAACTTTGTGGACGCTCTTTATAGGTTAATAAG
GCCGTATACAGCAGTTGTAACGATCGTAAGTGTAGTCGCGATGTCCCTTC
TTACAGTTAATAGCCTTTCAGATTTTTCCCCATTGTTCTTCATCAAAGTG
GTACAGGCTCTTATTGGAGGCATATTCATGCAAATGTATGTTAGTGGTTT
CAATCAAATTTGTGATATAGAACTCGACAAGGTTAACAAACAGTCTCTTC
CATTAGCGGCTGGAGAACTATCTATGAAAACTGCGATCGTCATCGCATCA
CTATCAGCTATCATGAGCTTATCGATTGGTTGGTTTGTTGGCTCCCCACC
ATTATTGTGGTGTCTTGTTTGGTGGTTTATTGTTGGGACTGCATATTCGG
CCAACGTGCTGCCTTATTTGCGATGGAAAAGGTTTCCTTTCACAGCAGCA
TTTTGCGCCATGACGTCTCGGGCACTAGTTCTTCCTATTGGATATTACTT
GCATATGCAGAATTCCATCCCGGGAGTATCTGCATTACTTTCAAGGCCAA
TATTATTTGCAGTCGCAATGCTCAGTGCATTTTCTTTATCAGCGATGTTC
TTTAAGGACATCCCTGATATTAAGGGAGATAGGATGCATGGAATCAAGTC
TCTAGCAATTAAACTGGGTGAAAAACGGGTGTATTGGATTTCCATTTCGA
TTATTGAAATTGCTTATATTGCTGCTGCATTTATTGGAGCAACTTCACCC
ATAAGCTGGAGCAAGTATGTAACGATTATCGGTCATCTTGGAATGGGATT
ACTACTTTGGGTACGAGCCAGATCAGTAGATCCGACGAACACGGTAGCCG
TTCAATCGATGTATATGTTCCTTATTAAGCTAGTATATGCAGAATACGGA
CTTATCTCGCTTGTACGCTGA.

In some embodiments, the polynucleotide comprises a nucleic acid sequence with at least 77%, at least 79%, at least 85%, at least 95%, or at least 99% homology or identity to SEQ ID NO: 23, or any value and range therebetween. Each possibility represents a separate embodiment of the invention. In some embodiments, the polynucleotide comprises a nucleic acid sequence with 77% to 100%, 85% to 100%, 90% to 100%, or 95% to 100% homology or identity to SEQ ID NO: 23. Each possibility represents a separate embodiment of the invention.

In some embodiments, the polynucleotide of the invention comprises 950 to 1,750 nucleotides. In some embodiments, the polynucleotide of the invention is 1,100 to 1,500 nucleotides long.

In some embodiments, 950 to 1,750 nucleotides comprises: at least 970 nucleotides, at least 1,000 nucleotides, at least 1,100 nucleotides, at least 1,150 nucleotides, at least 1,250 nucleotides, at least 1,400 nucleotides, at least 1,500 nucleotides, at least 1,600 nucleotides, or at least 1,730 nucleotides, or any value and range therebetween. Each possibility represents a separate embodiment of the invention. In some embodiments, 950 to 1,750 nucleotides comprises: 950 to 1,250 nucleotides, 1,100 to 1,350 nucleotides, 970 to 1,325 nucleotides, 1,150 to 1,400 nucleotides, or 1,170 to 1,490 nucleotides. Each possibility represents a separate embodiment of the invention.

In some embodiments, the polynucleotide comprises a plurality of polynucleotides. In some embodiments, the polynucleotide comprises a plurality of types of polynucleotides. As used herein, the term “plurality” comprises any integer equal to or greater than 2. In some embodiments, the polynucleotide comprises at least 2, at least 3, at least 4, at least 5, at least 6, at least 7, or 8 different nucleic acid sequences, or any value and range therebetween, wherein each of the different nucleic acid sequences is selected from SEQ ID Nos.: 1-11 and 23. Each possibility represents a separate embodiment of the invention. In some embodiments, the polynucleotide comprises 2-3, 2-4, 2-5, 2-8, 2-11, 3-7, 3-9, 3-11, 4-10, 4-11, 6-8, 6-11, 7-10, 7-11, 8-10, 9-11, or 10-11 different nucleic acid sequences, wherein each of the different nucleic acid sequences is selected from SEQ ID Nos.: 1-11 and 23.

In some embodiments, the polynucleotide is or comprises a plurality of polynucleotide molecules, wherein each of the plurality of the polynucleotide molecules comprises a different nucleic acid sequence, and wherein the different nucleic acid sequences are selected from SEQ ID Nos.: 1-11 and 23.

In some embodiments, the polynucleotide encodes a protein characterized by prenyl transferring activity. In some embodiments, the polynucleotide encodes a protein being a prenyltransferase (PT). In some embodiments, the PT is a PT derived from Helichrysum umbraculigerum. As used herein, the terms “prenyltransferase” and “PT” encompass any enzyme derived from H. umbraculigerum and having or characterized by being functional analog of the “geranylpyrophosphate:olivetolate geranyltransferase” or “GOT” of Cannabis sativa. In some embodiments, the GOT is GOT4 or CsGOT4.

As used herein, the terms “prenyltransferase” and “PT” are interchangeable, and refer to any peptide, polypeptide, or a protein, capable of transferring an allylic prenyl group to an acceptor molecule. In some embodiments, PT activity comprises cyclization. In some embodiments, PT activity comprises transferring an allylic prenyl group to an acceptor molecule.

According to some embodiments, there is provided an artificial nucleic acid molecule comprising the polynucleotide disclosed herein.

In some embodiments, the artificial vector comprises a plasmid. In some embodiments, the artificial vector comprises or is an agrobacterium comprising the artificial nucleic acid molecule. In some embodiments, the artificial vector is an expression vector. In some embodiments, the artificial vector is a plant expression vector. In some embodiments, the artificial vector is for use in expressing a PT encoding nucleic acid sequence as disclosed herein. In some embodiments, the artificial vector is for use in heterologous expression of a PT encoding nucleic acid sequence as disclosed herein in a cell, a tissue, or an organism.

Expressing of a polynucleotide within a cell is well known to one skilled in the art. It can be carried out by, among many methods, transfection, viral infection, or direct alteration of the cell's genome. In some embodiments, the polynucleotide is in an expression vector such as plasmid or viral vector. A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector, a virgaviridae viral vector, or a poxviral vector. The barley stripe mosaic virus (BSMV), the tobacco rattle virus and the cabbage leaf curl geminivirus (CbLCV) may also be used. The promoters may be active in plant cells. The promoters may be a viral promoter.

In some embodiments, the polynucleotide as disclosed herein is operably linked to a promoter. The term “operably linked” is intended to mean that the nucleotide sequence of interest is linked to the regulatory element or elements in a manner that allows for expression of the nucleotide sequence (e.g., in an in vitro transcription/translation system or in a host cell when the vector is introduced into the host cell). In some embodiments, the promoter is operably linked to the polynucleotide of the invention. In some embodiments, the promoter is a heterologous promoter. In some embodiments, the promoter is the endogenous promoter.

In some embodiments, the vector is introduced into the cell by standard methods including electroporation (e.g., as described in From et al., Proc. Natl. Acad. Sci. USA 82, 5824 (1985)), heat shock, infection by viral vectors, high velocity ballistic penetration by small particles with the nucleic acid either within the matrix of small beads or particles, or on the surface (Klein et al., Nature 327. 70-73 (1987)), such as biolistic use of coated particles, and needle-like particles, Agrobacterium Ti plasmids and/or the like. [096] The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins. The promoter may extend upstream or downstream of the transcriptional start site and may be any size ranging from a few base pairs to several kilo-bases.

In some embodiments, the polynucleotide is transcribed by RNA polymerase II (RNAP II and Pol II). RNAP II is an enzyme found in eukaryotic cells, known to catalyze the transcription of DNA to synthesize precursors of mRNA and most snRNA and microRNA.

In some embodiments, a plant expression vector is used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3: 1671-1680 (1984); and Brogli et al., Science 224:838-843 (1984)] or heat shock promoters, e.g., soybean hspl7.5-E or hspl7.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as systemic infection and targeting specificity, are used for in vivo expression. In one embodiment, systemic infection is inherent in the life cycle of, for example, the retrovirus and is the process by which a single infected cell produces many progeny virions that infect neighboring cells. In one embodiment, the result is that a large area becomes rapidly infected, most of which was not initially infected by the original viral particles. In one embodiment, viral vectors are produced that are unable to spread systemically. In one embodiment, this characteristic can be useful if the desired purpose is to introduce a specified gene into only a localized number of targeted cells.

In some embodiments, plant viral vectors are used. In some embodiments, a wild-type virus is used. In some embodiments, a deconstructed virus such as are known in the art is used. In some embodiments, Agrobacterium is used to introduce the vector of the invention into a plant.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 19866] and include, for example, stable or transient transfection, lipofection, electroporation, agrobacterium Ti plasmids and infection with recombinant viral vectors. In addition, see U.S. Pat. Nos. 5,464,764 and 5,487,992 for positive-negative selection methods.

It will be appreciated that other than containing the necessary elements for the transcription and translation of the inserted coding sequence (encoding the polypeptide), the expression construct of the present invention can also include sequences engineered to optimize stability, production, purification, yield, or activity of the expressed polypeptide.

In some embodiments, the artificial vector comprises a polynucleotide encoding a protein comprising an amino acid sequence as described herein.

According to some embodiments, there is provided a protein encoded by: (a) the polynucleotide disclosed herein; (b) the artificial vector disclosed herein; or the plasmid or agrobacterium disclosed herein.

In some embodiments, the protein is encoded by a polynucleotide comprising or consisting of SEQ ID Nos.: 1-11, and 23.

In some embodiments, the protein comprises an amino acid sequence with at least 92%, at least 93%, at least 95%, at least 97%, or at least 99% homology or identity to any one of SEQ ID Nos.: 12-22, and 24.

In some embodiments, the protein is an isolated protein.

As used herein, the terms “peptide”, “polypeptide” and “protein” are interchangeable and refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides, polypeptides and proteins described have modifications rendering them more stable while in the organism or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid.

As used herein, the terms “isolated protein” refers to a protein that is essentially free from contaminating cellular components, such as carbohydrate, lipid, or other proteinaceous impurities associated with the nucleic acid in nature. Typically, a preparation of an isolated protein contains the protein in a highly purified form, e.g., at least about 80% pure, at least about 90% pure, at least about 95% pure, greater than 95% pure, or greater than 99% pure. In some embodiments, the isolated protein is a synthesized protein. Synthesis of protein is well known in the art and may be performed, for example, by heterologous expression in a transformed cell, such as exemplified herein.

In some embodiments, the protein comprises or consists of the amino acid sequence:

(SEQ ID NO: 12)
MELSLSSSSSSSLPQLHTHPSSSSSSSHYIKKSPFFINKFNNHTKCKFHN
SSALRTNFFYTTITKTSSSRFVLNKNPNQFSVKACSQVGSAGSDPALNKV
ADFKDAFWRFLRPHTIRGTALGSVSLVTRALLENPNLIRWSLLLKAFSGL
VALICGNGYIVGINQIYDIGIDKVNKPYLPIAAGDLSVQSAWFLVLAFAM
VGVIIVGMNFGPFITSLYSLGLFLGTIYSVPPLRMKRFPVVAFLIIATVR
GFLLNFGVYYAVRAALGLTFQWSSAVAFITTFVTLFALVIAITKDLPDVE
GDRKFQISTFATKLGVRNIALLGSGLLLINYIGSIVAALYMPQAFRSSLM
IPLHTILASCLIYQAWILERANYTQEAIAGYYRFVWNLFYSEYIIFPFI.

In some embodiments, the protein comprises an amino acid sequence with at least 82%, at least 85%, at least 90%, or at least 99% homology or identity to SEQ ID NO: 12, or any value and range therebetween. Each possibility represents a separate embodiment of the invention. In some embodiments, the protein comprises an amino acid sequence with 82% to 100%, 85% to 100%, 90% to 100%, or 95% to 100% homology or identity to SEQ ID NO: 12. Each possibility represents a separate embodiment of the invention.

In some embodiments, the protein comprises or consists of the amino acid sequence:

(SEQ ID NO: 13)
MATMASSLLNPLSCSIKPNSNRLPLPTPISLSRSCRRLTIKATETDANEV
KPKAPEKAPAASGSGFNQILGIKGAKQETNKWKIRVQLTKPVTWPPLIWG
VVCGAAASGNFQWTVEDVAKSIVCMLMSGPFLTGYTQTINDWYDRDIDAI
NEPYRPIPSGAISENEVITQIWVLLLGGIGLAGILDVWAGHKSPTIFYLA
LGGSLLSYIYSAPPLKLKQNGWIGNFALGASYISLPWWAGQALFGTLTPD
IVVLTLLYSIAGLGIAIVNDFKSVEGDRKMGLQSLPVAFGEETAKWICVG
AIDITQLSIAGYLLGSGKPYYALALVGLIVPQIFFQFKYFLKDPVKYDVK
YQASAQPFLILGLLVTALATSH.

In some embodiments, the protein comprises an amino acid sequence with at least 92%, at least 93%, at least 95%, at least 97%, at least 98%, or at least 99% homology or identity to SEQ ID NO: 13, or any value and range therebetween. Each possibility represents a separate embodiment of the invention. In some embodiments, the protein comprises an amino acid sequence with 92% to 100%, 93% to 100%, 95% to 100%, or 97% to 100% homology or identity to SEQ ID NO: 13. Each possibility represents a separate embodiment of the invention.

In some embodiments, the protein comprises or consists of the amino acid sequence:

(SEQ ID NO: 14)
MKSLIIGSFSNKVSCYSPSLPDSSSSLIPTGCYHVSLRTFQRNRAIQAQS
SLVRCNIGKFNETLLLSRKRSTKHVACAVSEQPIEPDATNPQSSLPNALD
AFYRFSRPHTVIGTALSIVSVSLLAVQKLSDFSPLFFIGVFEAIVAAFFM
NIYIVGLNQLSDIEIDKVNKPYLPLASGEYSVQTGIIIVSSFAVMSFWLG
WIVGSWPLFWALFISFLLGTAYSINIPMLRWKRFALVAAMCILAVRAIIV
QVAFYLHIQTFVYGRLAVFPKPVIFATGFMSFFSVVIALFKDIPDIVGDK
IFGIQSFTVRMGQKRVFWICILLLEIAYGVAILVGASSPFLWSRYITVLG
HAILGLILWGRAKSTDLESKSAITSFYMFIWQLFYAEYLLIPLVR.

In some embodiments, the protein comprises an amino acid sequence with at least 89%, at least 90%, at least 95%, or at least 99% homology or identity to SEQ ID NO: 14, or any value and range therebetween. Each possibility represents a separate embodiment of the invention. In some embodiments, the protein comprises an amino acid sequence with 89% to 100%, 92% to 100%, 94% to 100%, or 96% to 100% homology or identity to SEQ ID NO: 14. Each possibility represents a separate embodiment of the invention.

In some embodiments, the protein comprises or consists of the amino acid sequence:

(SEQ ID NO: 15)
MELSLSSSSSSSLPQLHTHPSSSSSSSHYIKKSPFFINKFNNHTKCKFHN
SSALRTNFFYTTITKTSSSRFVLNKNPNQFSVKACSQVGSAGSDPALNKV
ADFKDAFWRFLRPHTIRGTALGSVSLVTRALLENPNLIRWSLLLKAFSGL
VALICGNGYIVGINQIYDIGIDKVNKPYLPIAAGDLSVQSAWFLVLAFAM
VGVIIVGMNFGPFITSLYSLGLFLGTIYSVPPLRMKRFPVVAFLIIATVR
GFLLNFGVYYAVRAALGLTFQWSSAVAFITTFVTLFALVIAITKDLPDVE
GDRKFQISTFATKLGVRNIALLGSGLLLINYIGSIVAALYMPQAFRSSLM
IPLHTILASCLIYQAWILERANYTQRSQYFDMSSCRRR.

In some embodiments, the protein comprises an amino acid sequence with at least 81%, at least 85%, at least 90%, at least 95%, at least 97%, or at least 99% homology or identity to SEQ ID NO: 15, or any value and range therebetween. Each possibility represents a separate embodiment of the invention. In some embodiments, the protein comprises an amino acid sequence with 81% to 100%, 85% to 100%, 88% to 100%, or 93% to 100% homology or identity to SEQ ID NO: 15. Each possibility represents a separate embodiment of the invention.

In some embodiments, the protein comprises or consists of the amino acid sequence:

(SEQ ID NO: 16)
MELSLSSSSSSSLPQLHTHPSSSSSSSHYIKKSPFFINKFNNHTKCKFHN
SSALRTNFFYTTITKTSSSRFVLNKNPNQFSVKACSQVGSAGSDPALNKV
ADFKDAFWRFLRPHTIRGTALGSVSLVTRALLENPNLIRWSLLLKAFSGL
VALICGNGYIVGINQIYDIGIDKVNKPYLPIAAGDLSVQSAWFLVLAFAM
VGVIIVGMNFGPFITSLYSLGLFLGTIYSVPPLRMKRFPVVAFLIIATVR
GFLLNFGVYYAVRAALGLTFQWSSAVAFITTFVTLFALVIAITKDLPDVE
GDRKFQISTFATKLGVRNIALLGSGLLLINYIGSIVAALYMPQVKTTSID
HYRPYSFLVDLPGQNGITLAA.

In some embodiments, the protein comprises an amino acid sequence with at least 81%, at least 85%, at least 90%, at least 95%, at least 97%, or at least 99% homology or identity to SEQ ID NO: 16, or any value and range therebetween. Each possibility represents a separate embodiment of the invention. In some embodiments, the protein comprises an amino acid sequence with 81% to 100%, 85% to 100%, 90% to 100%, or 95% to 100% homology or identity to SEQ ID NO: 16. Each possibility represents a separate embodiment of the invention.

In some embodiments, the protein comprises or consists of the amino acid sequence:

(SEQ ID NO: 17)
MATMASSLLNPLSCSIKPNSNRLPLPLPIPISLSRSCRRLTIKATETDAN
EVKPKAPEKAPAASGSGFNQILGIKGAKQETNKWKIRVQLTKPVTWPPLI
WGVVCGAAASGNFQWTVEDVAKSIVCMLMSGPFLTGYTQTINDWYDRDID
AINEPYRPIPSGAISENEVITQIWVLLLGGIGLAGILDVWAGHKSPTIFY
LALGGSLLSYIYSAPPLKLKQNGWIGNFALGASYISLPWWAGQALFGTLT
PDIVVLTLLYSIAGLGIAIVNDFKSVEGDRKMGLQSLPVAFGEETAKWIC
VGAIDITQLSIAGYLLGSGKPYYALALVGLIVPQIFFQFKYFLKDPVKYD
VKYQASAQPFLILGLLVTALATSH.

In some embodiments, the protein comprises an amino acid sequence with at least 92%, at least 93%, at least 95%, at least 97%, at least 98%, or at least 99% homology or identity to SEQ ID NO: 17, or any value and range therebetween. Each possibility represents a separate embodiment of the invention. In some embodiments, the protein comprises an amino acid sequence with 92% to 100%, 93% to 100%, 96% to 100%, or 98% to 100% homology or identity to SEQ ID NO: 17. Each possibility represents a separate embodiment of the invention.

In some embodiments, the protein comprises or consists of the amino acid sequence:

(SEQ ID NO: 18)
MASLAIGSLGSPSSRQCSSPVASSSSFAIGSQIASKFLRISKFDKTKNSP
LTLQQKHINKSIDQSFFEPLPLHKINKDKFKLYATSTNNPQFDATHDLKT
PEVSIINFVDALYRLIRPYTAVVTIVSVVAMSLLTVNSLSDFSPLFFIKV
VQALIGGIFMQMYVSGFNQICDIELDKVNKQSLPLAAGELSMKTAIVIAS
LSAIMSLSIGWFVGSPPLLWCLVWWFIVGTAYSANVLPYLRWKRFPFTAA
FCAMTSRALVLPIGYYLHMQNSIPGVSALLSRPILFAVAMLSAFSLSAMF
FKDIPDIKGDRMHGIKSLAIKLGEKRVYWISISIIEIAYIAAAFIGATSP
ISWSKYVTIIGHLGMGLLLWVRARSVDPTNTVAVQSMYMFLIKLVYAEYG
LISLVR.

In some embodiments, the protein comprises an amino acid sequence with at least 71%, at least 75%, at least 80%, at least 85%, at least 90%, at least 95%, at least 97%, or at least 99% homology or identity to SEQ ID NO: 18, or any value and range therebetween. Each possibility represents a separate embodiment of the invention. In some embodiments, the protein comprises an amino acid sequence with 71% to 100%, 75% to 100%, 80% to 100%, or 90% to 100% homology or identity to SEQ ID NO: 18. Each possibility represents a separate embodiment of the invention.

In some embodiments, the protein comprises or consists of the amino acid sequence:

(SEQ ID NO: 19)
MKSLIIGSFSNKVSCYSPSLPDSSSSLIPTGCYHVSLRTFQRNRAIQAQS
SLVRCNIGKFNETLLLSRKRSTKHVACAVSEQPIEPDATNPQSSLPNALD
AFYRFSRPHTVIGTALSIVSVSLLAVQKLSDFSPLFFIGVFEAIVAAFFM
NIYIVGLNQLSDIEIDKVNKPYLPLASGEYSVQTGIIIVSSFAVMSFWLG
WIVGSWPLFWALFISFLLGTAYSINIPMLRWKRFALVAAMCILAVRAIIV
QVAFYLHIQTFVYGRLAVFPKPVIFATGFMSFFSVVIALFKDIPDIVGDK
IFGIQSFTVRMGQKRVFWICILLLEIAYGVAILVGASSPFLWSRYITVLG
HAILGLILWGRAKSTDLESKSAITSFYMFIWQLFYAEYLLIPLVR.

In some embodiments, the protein comprises an amino acid sequence with at least 89%, at least 90%, at least 95%, or at least 99% homology or identity to SEQ ID NO: 19, or any value and range therebetween. Each possibility represents a separate embodiment of the invention. In some embodiments, the protein comprises an amino acid sequence with 89% to 100%, 92% to 100%, 95% to 100%, or 97% to 100% homology or identity to SEQ ID NO: 19. Each possibility represents a separate embodiment of the invention.

In some embodiments, the protein comprises or consists of the amino acid sequence:

(SEQ ID NO: 20)
MLIHHEHFLTTGFESSNDRAAYSINFSKQHHLHMASIATGSLCRPTSHQF
SIPVASSSSFATGSQFASKFLHISISAKKSSLTLQQRHIHKNIDQSFLKP
LALQKLNKDKFKLNGTSPDNPQFDATHDLKTQIESTINFVDVLYRLLRPY
ALLQMGLCVVTMSLLTVESLSDFSPLFFVKVAQALIGGIFMQMYVNGFNQ
ICDIELDKVNKPSLPLASGELSKTTTIVVSSLSAITSLSIGWFVGSPPLL
WSLVVWFIAGTTYSANLPYLRWKRFPFTNMFCNLTMALVVPIGTYLHMEN
SIHGVSTLLSRPLLFTVAMCTVFPVSIILFKDIPDIKGDRMHGMKSLAII
LGEKRTYWICIWILEITYIAAAFFGATSPISWSKYVTIISHLGMGFLLWL
RSKSVDVKNTVAVQSMYMFLWKLLYAEYGLILLVR.

In some embodiments, the protein comprises an amino acid sequence with at least 68%, at least 75%, at least 80%, at least 855, at least 90%, at least 95%, or at least 99% homology or identity to SEQ ID NO: 20, or any value and range therebetween. Each possibility represents a separate embodiment of the invention. In some embodiments, the protein comprises an amino acid sequence with 68% to 100%, 75% to 100%, 80% to 100%, or 90% to 100% homology or identity to SEQ ID NO: 20. Each possibility represents a separate embodiment of the invention.

In some embodiments, the protein comprises or consists of the amino acid sequence:

(SEQ ID NO: 21)
MFIHHEQFLTTGFESSNDRAAYSINFLKQHHLHMVSIATGSLCRPTSHRF
SIPVASSSSFATGSQFASISAKKSSLTLKQRHTHKNIDQSFFKPLALQKM
NKGKFKLNATSPDNSQLDATHDLKTQIESIINFVDVLYRLIRPYVVLGMG
VTIVTMCLLTVDSLSDFSPLFFVKVAQALIGSIFMAMYVNSFNEICDIEL
DKVNKPSLPLASGELSMTTAIVVSSLSAIMSLSIGWFVGSPPLLWSLVVW
FILGTAYSANLPYLRWKRFPLTTLSSALTMGALVIPIGNYMHMENSIRGV
TTLLSRPLLFAVAMCAAFHVSTILFKDIPDIKGDRMHGMKSLAIKLGEKR
MYWICIWILEIAYIAAAFFGATSPISWSKYVTIISHLGMGFLLWLRSKS
VDVKNTVAVQSMYMFLWKLFYVEHGLILLVR.

In some embodiments, the protein comprises an amino acid sequence with at least 66%, at least 75%, at least 80%, at least 855, at least 90%, at least 95%, or at least 99% homology or identity to SEQ ID NO: 21, or any value and range therebetween. Each possibility represents a separate embodiment of the invention. In some embodiments, the protein comprises an amino acid sequence with 66% to 100%, 75% to 100%, 85% to 100%, or 90% to 100% homology or identity to SEQ ID NO: 21. Each possibility represents a separate embodiment of the invention.

In some embodiments, the protein comprises or consists of the amino acid sequence:

(SEQ ID NO: 22)
MASIATGSLCRPTSHRFSIHVASSSSFATGSQFASKILQISISAKKSSLT
LQQRHIHKNIDQSFFKPLALQKMNKDKFKLNATSPDNPQFDATRDLKTQI
ESIIKFVDVLYRLLRPYAILEMGLSVVTMSLLTVESLSDFSPLFFVKVAQ
ALIGGIFMQMYVNGFNQICDIELDKVNKPSLPLASGELSTTTTIVVSSLS
AIMSLSIGWFVGSPPLLWSLVVWFIVGTTYSTNLPYLRWKRFPFTAMFCN
LTRALVVPIGTYLHMKNSIHEVSTLLSRPLLFAVAMCTVFPISIILFKDI
PDIKGDRMHGMKSLAIILGEERTYWICIWILEIAYIAAAFFGATSPISWS
KYVMIISHLGMGFLLWLRSKSVDVKNTVAVQSMYMFLWKLLYAEYGLILL
VR.

In some embodiments, the protein comprises an amino acid sequence with at least 68%, at least 75%, at least 80%, at least 855, at least 90%, at least 95%, or at least 99% homology or identity to SEQ ID NO: 22, or any value and range therebetween. Each possibility represents a separate embodiment of the invention. In some embodiments, the protein comprises an amino acid sequence with 68% to 100%, 75% to 100%, 85% to 100%, or 95% to 100% homology or identity to SEQ ID NO: 22. Each possibility represents a separate embodiment of the invention.

In some embodiments, the protein comprises or consists of the amino acid sequence:

(SEQ ID NO: 24)
MASLAIGSLGSPSSRQCSSPVASSSSFAIGSQIASKFLRISKFDKTKNSP
LALQQKHINKSIDQSFFEPLPLHKINKDKFKLYATSTNNPQFDATHDLKT
PEVSIINFVDALYRLIRPYTAVVTIVSVVAMSLLTVNSLSDFSPLFFIKV
VQALIGGIFMQMYVSGFNQICDIELDKVNKQSLPLAAGELSMKTAIVIAS
LSAIMSLSIGWFVGSPPLLWCLVWWFIVGTAYSANVLPYLRWKRFPFTAA
FCAMTSRALVLPIGYYLHMQNSIPGVSALLSRPILFAVAMLSAFSLSAMF
FKDIPDIKGDRMHGIKSLAIKLGEKRVYWISISIIEIAYIAAAFIGATSP
ISWSKYVTIIGHLGMGLLLWVRARSVDPTNTVAVQSMYMFLIKLVYAEYG
LISLVR.

In some embodiments, the protein comprises an amino acid sequence with at least 71%, at least 75%, at least 80%, at least 85%, at least 90%, at least 95%, at least 97%, or at least 99% homology or identity to SEQ ID NO: 24, or any value and range therebetween. Each possibility represents a separate embodiment of the invention. In some embodiments, the protein comprises an amino acid sequence with 71% to 100%, 80% to 100%, 90% to 100%, or 95% to 100% homology or identity to SEQ ID NO: 24. Each possibility represents a separate embodiment of the invention.

The terms “homology” or “identity”, as used interchangeably herein, refer to sequence identity between two amino acid sequences or two nucleic acid sequences, with identity being a stricter comparison. The phrases “percent identity or homology” and “% identity or homology” refer to the percentage of sequence identity found in a comparison of two or more amino acid sequences or nucleic acid sequences. Two or more sequences can be anywhere from 0-100% identical, or any value there between. Identity can be determined by comparing a position in each sequence that can be aligned for purposes of comparison to a reference sequence. When a position in the compared sequence is occupied by the same nucleotide base or amino acid, then the molecules are identical at that position. A degree of identity of amino acid sequences is a function of the number of identical amino acids at positions shared by the amino acid sequences. A degree of identity between nucleic acid sequences is a function of the number of identical or matching nucleotides at positions shared by the nucleic acid sequences. A degree of homology of amino acid sequences is a function of the number of amino acids at positions shared by the polypeptide sequences.

The following is a non-limiting example for calculating homology or sequence identity between two sequences (the terms are used interchangeably herein). The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frame shift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences.

In some embodiments, % homology or identity as described herein are calculated or determined using the basic local alignment search tool (BLAST). In some embodiments, % homology or identity as described herein are calculated or determined using Blossum 62 scoring matrix.

In some embodiments, the protein comprises or is characterized by prenyl transferring activity, as described herein. In some embodiments, the protein is characterized by being capable of transferring a prenyl group to a substrate molecule. In some embodiments, the protein is characterized by being capable of transferring an allylic prenyl group to an acceptor molecule. In some embodiments, the protein is a prenyl diphosphate synthase. In some embodiments, the protein is a trans-prenyltranferase. In some embodiments, the protein is a cis-prenyltransferase.

In some embodiments, the prenyl group is selected from: dimethylallyl diphosphate, geranyl diphosphate, farnesyl diphosphate, or geranylgeranyl diphosphate.

In some embodiments, the substrate molecule is represented by Formula I:

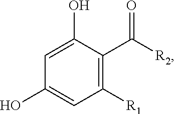

wherein: (i) R₁ is selected from: C1-C8 alkyl, an alpha-unsaturated phenylalkyl carboxylic acid, or an alpha saturated phenylalkyl carboxylic acid; and R₂ is OH; or (ii) R₁ is OH and R₂ is selected from: C1-C8 alkyl, an alpha-unsaturated phenylalkyl carboxylic acid, or an alpha saturated phenylalkyl carboxylic acid.

In some embodiments, an alpha-unsaturated phenylalkyl carboxylic acid comprises cinnamic acid or a derivative thereof.

In some embodiments, a cinnamic acid derivative is or comprises a hydroxylated derivative of cinnamic acid.

In some embodiments, a hydroxylated derivative of cinnamic acid is or comprises coumaric acid.

According to some embodiments, there is provided a transgenic cell comprising: (a) the polynucleotide disclosed herein; (b) the artificial nucleic acid molecule disclosed herein; (c) the plasmid or agrobacterium disclosed herein; (d) the isolated protein disclosed herein; or any combination thereof.

As used herein, the term “transgenic cell” refers to any cell that has undergone human manipulation on the genomic or gene level. In some embodiments, the transgenic cell has had exogenous polynucleotide, such as an isolated DNA molecule as disclosed herein, introduced into it. In some embodiments, a transgenic cell comprises a cell that has an artificial vector introduced into it. In some embodiments, a transgenic cell is a cell which has undergone genome mutation or modification. In some embodiments, a transgenic cell is a cell that has undergone CRISPR genome editing. In some embodiments, a transgenic cell is a cell that has undergone targeted mutation of at least one base pair of its genome. In some embodiments, the exogenous polynucleotide (e.g., the isolated DNA molecule disclosed herein) or vector is stably integrated into the cell. In some embodiments, the transgenic cell expresses a polynucleotide of the invention. In some embodiments, the transgenic cell expresses a vector of the invention. In some embodiments, the transgenic cell expresses a protein of the invention. In some embodiments, the transgenic cell, is a cell that is devoid of a polynucleotide of the invention that has been transformed or genetically modified to include the polynucleotide of the invention. In some embodiments, CRISPR technology is used to modify the genome of the cell, as described herein.

In some embodiments, the cell is a unicellular organism, a cell of a multicellular organism, and a cell in a culture.

In some embodiments, a unicellular organism comprises a fungus or a bacterium.

In some embodiments, the fungus is a yeast cell.

In some embodiments, the cell is an insect cell. In some embodiments, the cell comprises an insect cell line.

Types of insect cell lines suitable for transformation and/or heterologous expression are common and would be apparent to one of ordinary skill in the art. Non-limiting examples of such insect cell lines include, but are not limited to, Sf-9 cells, SR+ Schneider cells, S2 cells, and others.

According to some embodiments, there is provided an extract derived from a transgenic cell disclosed herein, or any fraction thereof.

In some embodiments, the extract comprises the polynucleotide of the invention, an isolated DNA molecule as disclosed herein, an isolated protein as disclosed herein, or any combination thereof.

According to some embodiments, there is provided a homogenate, lysate, extract, derived from a transgenic cell disclosed herein, any combination thereof, or any fraction thereof.

Methods and/or means for extracting, lysing, homogenizing, fractionating, or any combination thereof, a cell or a culture of same, are common and would be apparent to one of ordinary skill in the art of cell biology and biochemistry. Non-limiting examples include, but are not limited to, pressure lysis (e.g., such as using a French press), enzymatic lysis, soluble-insoluble phase separation (such for obtaining a supernatant and a pellet), detergent-based lysis, solvent (e.g., polar or nonpolar solvent), liquid chromatography mass spectrometry, or others.

According to some embodiments, there is provided a transgenic plant, a transgenic plant tissue or a plant part. In some embodiments, there is provided a transgenic plant, or any portion, seed, tissue or organ thereof, comprising at least one transgenic plant cell of the invention. In some embodiments, the transgenic plant, transgenic plant tissue or plant part, comprises: (a) the polynucleotide disclosed herein; (b) the artificial disclosed herein; (c) the plasmid or agrobacterium disclosed herein; (d) the isolated protein of the invention; (e) the transgenic cell disclosed herein; or any combination thereof.

In some embodiments, the transgenic plant, transgenic plant tissue, or plant part consists of transgenic plant cells of the invention. In some embodiments, the transgenic plant, transgenic plant tissue, or plant part comprises at least: 20%, 25%, 30%, 35%, 40%, 45%, 50%, 55%, 60%, 65%, 70%, 75%, 80%, 85%, 90%, 95%, 97%, or 99% transgenic cells of the invention, or any value and range therebetween. Each possibility represents a separate embodiment of the invention. In some embodiments, the transgenic plant, transgenic plant tissue, or plant part comprises 20%-50%, 20%-60%, 20%-70%, 20%-80%, 20%-90%, or 20%-100% transgenic cells of the invention. Each possibility represents a separate embodiment of the invention.

In some embodiments, the transgenic plant, transgenic plant tissue, or plant part is or derived from a Cannabis sativa plant. In some embodiments, the transgenic plant is a C. sativa plant.

In some embodiments, the transgenic plant, transgenic plant tissue, or plant part is or derived from hemp. In some embodiments, C. sativa comprises or is hemp.

According to some embodiments, there is provided a composition comprising any one of the herein disclosed: (a) polynucleotide of the invention (for example, an isolated DNA molecule); (b) artificial vector; (c) plasmid or agrobacterium; (d) isolated protein of the invention; (e) transgenic cell; (f) extract; (g) transgenic plant tissue or plant part; and (h) any combination of (a) to (g), and an acceptable carrier.

As used herein, the term “carrier”, “excipient”, or “adjuvant” refers to any component of a composition, e.g., pharmaceutical or nutraceutical, that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier (e.g. carbomer, hydroxypropyl cellulose, sodium lauryl sulfate) as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers, and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers, and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

### Methods of Synthesis

According to some embodiments, there is provided a method for synthesizing a compound represented by Formula II:

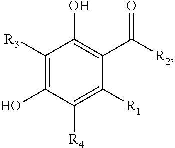

wherein: (i) R₁ is selected from: C1-C8 alkyl, an alpha-unsaturated phenylalkyl carboxylic acid, or an alpha saturated phenylalkyl carboxylic acid, and R₂ is OH; or (ii) R₁ is OH and R₂ is selected from: C1-C8 alkyl, an alpha-unsaturated phenylalkyl carboxylic acid, or an alpha saturated phenylalkyl carboxylic acid, wherein R₃ is a prenyl group, and wherein R₄ is hydrogen or a prenyl group.

According to some embodiments, the method comprises the steps: (a) providing a cell comprising an artificial vector comprising a nucleic acid sequence having at least 91%, at least 92%, at least 93%, at least 94%, at least 95%, at least 97%, or at least 99% homology or identity to any one of SEQ ID Nos.: 1-11 and 23, or any combination thereof, or any value and range therebetween; and (b) culturing the cell from step (a) such that a protein encoded by the artificial vector is expressed, thereby synthesizing the compound represented by Formula II. Each possibility represents a separate embodiment of the invention.

According to some embodiments, the method comprises contacting a substrate molecule represented by Formula I:

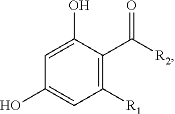

wherein: (i) R₁ is selected from: C1-C8 alkyl, an alpha-unsaturated phenylalkyl carboxylic acid, or an alpha saturated phenylalkyl carboxylic acid; and R₂ is OH; or (ii) R₁ is OH and R₂ is selected from: C1-C8 alkyl, an alpha-unsaturated phenylalkyl carboxylic acid, or an alpha saturated phenylalkyl carboxylic acid, with an effective amount of a protein comprising an amino acid sequence with at least 92%, at least 93%, at least 95%, at least 99%, or 100% homology or identity to any one of SEQ ID Nos.: 12-22 and 24, or any value and range therebetween, thereby synthesizing the compound represented by Formula II. Each possibility represents a separate embodiment of the invention.

According to some embodiments, there is provided a method for obtaining an extract from a transgenic cell or a transfected cell.

In some embodiments, the method comprises culturing a transgenic cell or a transfected cell in a medium and extracting the transgenic cell or the transfected cell.

In some embodiments, the method comprises the steps: (a) culturing a transgenic cell or a transfected cell in a medium; and (b) extracting the transgenic cell or the transfected cell, thereby obtaining an extract from the transgenic cell or the transfected cell.

In some embodiments, the transgenic cell or the transfected cell comprises an artificial vector comprising a nucleic acid sequence having at least 91%, at least 93%, at least 95%, at least 97%, at least 99%, or 100% homology or identity to any one of SEQ ID Nos.: 1-11 and 23, or any combination thereof, or any value and range therebetween. Each possibility represents a separate embodiment of the invention.

In some embodiments, the transgenic cell or the transfected cell comprises the polynucleotide of the invention or a plurality thereof, as disclosed herein.

In some embodiments, the transgenic cell or the transfected cell comprises the artificial nucleic acid molecule or vector as disclosed herein.

In some embodiments, the cell is a transgenic cell, or a cell transfected with an isolated DNA molecule as disclosed herein.

In some embodiments, the culturing comprises supplementing the cell with an effective amount of a substrate molecule represented by Formula I. In some embodiments, the supplementing is via the growth or culture medium wherein the cell is cultured.

In some embodiments, the substrate molecule is selected from: a resorcinoid precursor, a stilbene acid precursor, an acyl phloroglucinoid precursor, or a chalcone precursor.

In some embodiments, the substrate molecule is represented by a formula selected from:

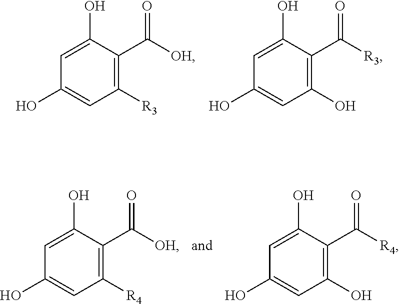

wherein R₃ is C1-C8 alkyl, and wherein R₄ is an alpha-unsaturated phenylalkyl carboxylic acid, or an alpha saturated phenylalkyl carboxylic acid.

In some embodiments, the substrate molecule is selected from:

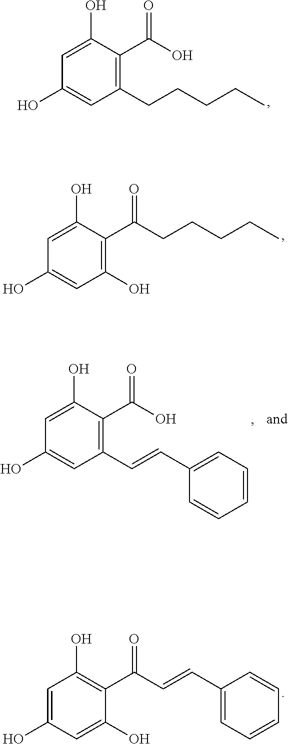 [SMILES: CCCCCc1cc(O)cc(O)c1C(=O)O ; CCCCCC(=O)c1c(O)cc(O)cc1O ; O=C(O)c1c(O)cc(O)cc1/C=C/c1ccccc1 ; O=C(/C=C/c1ccccc1)c1c(O)cc(O)cc1O]

In some embodiments, the substrate molecule is:

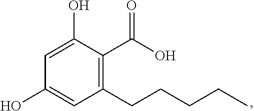 [SMILES: CCCCCc1cc(O)cc(O)c1C(=O)O]

In some embodiments, the compound represented by Formula II is selected from: a cannabinoid, an amorfrutin, an acyl phlorogluconoid, or a prenyl chalcone.

In some embodiments, the compound represented by Formula II is selected from:

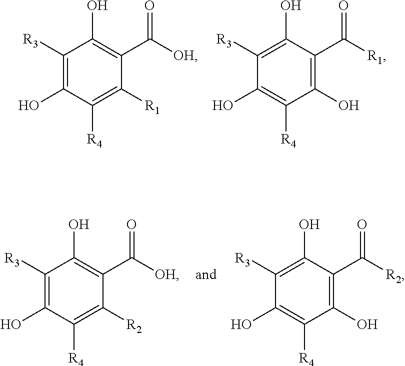

wherein: R₁ is C1-C8 alkyl, R₂ is an alpha-unsaturated phenylalkyl carboxylic acid or an alpha saturated phenylalkyl carboxylic acid, R₃ is a prenyl group, and R₄ is hydrogen or a prenyl group.

In some embodiments, the prenyl group is selected from: dimethylallyl diphosphate, geranyl diphosphate, farnesyl diphosphate, or geranylgeranyl diphosphate.

In some embodiments, the compound represented by Formula II is selected from:

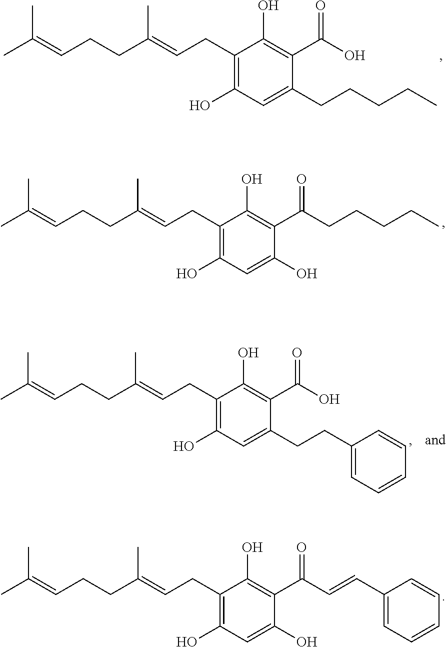

In some embodiments, the compound is:

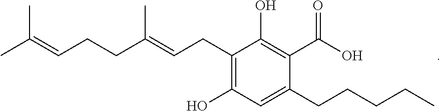 [SMILES: CCCCCc1cc(O)c(C/C=C(\C)CCC=C(C)C)c(O)c1C(=O)O]

In some embodiments, the method further comprises a step preceding step (a), comprising introducing or transfecting the cell with the artificial nucleic acid molecule or vector, disclosed herein.

Method for introducing or transfecting a cell with an artificial nucleic acid molecule or vector are common and would be apparent to one of ordinary skill in the art.

In some embodiments, introducing or transfecting comprises transferring an artificial nucleic acid molecule or vector comprising the polynucleotide disclosed herein into a cell; or modifying the genome of a cell to include the polynucleotide disclosed herein. In some embodiments, the transferring comprises transfection. In some embodiments, the transferring comprises transformation. In some embodiments, the transferring comprises lipofection. In some embodiments, the transferring comprises nucleofection. In some embodiments, the transferring comprises viral infection.

As used herein, the terms “transfecting” and “introducing” are interchangeable.

In some embodiments, the contacting is in a cell-free system.

Types of suitable cell-free systems for utilizing any one of: the polynucleotide of the invention or a plurality thereof, as disclosed herein, and the isolated protein of the invention, or a plurality thereof, would be apparent to one of ordinary skill in the art.

In some embodiments, the method further comprises a step preceding step (b), comprising separating the cultured transgenic cell or the cultured transfected cell from the medium.

Method for separating cell from a medium are common and may include, but not limited to, centrifugation, ultracentrifugation, or other, as would be apparent to one of ordinary skill in the art.

According to some embodiments, there is provided an extract of a transgenic cell or a transfected cell obtained according to the herein disclosed method.

According to some embodiments, there is provided a medium or a portion thereof separated from a cultured transgenic cell or a cultured transfected cell, obtained according to the herein disclosed method.

According to some embodiments, there is provided a composition comprising: (a) the extract disclosed herein; (b) the medium disclosed herein or a portion thereof; or (c) any combination of (a) and (b), and an acceptable carrier, as described herein.

In some embodiments, a portion comprises a fraction or a plurality thereof.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” when combined with a value refers to plus and minus 10% of the reference value. For example, a length of about 1,000 nanometers (nm) refers to a length of 1,000 nm±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### UPLC-qTOF Analysis of Cannabigerolic Acid (CBGA)

Fresh samples of six different tissues: young leaves, old leaves, florets and receptacle of flowers, stem, and root were collected from a plant at the flowering stage. Florets and the receptacle of flowers were detached using a scalpel and extracted separately. All the tissues were flash-frozen in liquid N₂, ground in a mortar to a fine powder, and extracted as previously described with 1 ml ethanol.

Samples were analyzed using a high-resolution ultrahigh-performance liquid chromatography-tandem quadrupole time-of-flight (UPLC-qTOF) system comprised of a UPLC (Waters Acquity) with a diode array detector connected either to a XEVO G2-S QTof (Waters) or to Synapt HDMS (Waters). The chromatographic separation of compounds was performed on a 100 mm×2.1 mm i.d. (internal diameter), 1.7 μm UPLC BEH C18 column (Waters Acquity). The mobile phase consisted of 0.1% formic acid in acetonitrile:water (5:95, v/v; phase A) and 0.1% formic acid in acetonitrile (phase B). The flow rate was 0.3 ml min⁻¹, and the column temperature was kept at 35° C. Plant extracts were analyzed using a 29 min multistep gradient method: initial conditions were 40% B for 1 min, raised to 100% B until 23 min, held at 100% B for 3.8 min, decreased to 40% B until 27 min, and held at 40% B until 29 min for re-equilibration of the system. Products form enzymatic assays were analyzed with a shorter second step (B was raised from 40% to 100% in 13 min). Electrospray ionization (ESI) was used in negative ionization with an m/z range of 50-1,000 Da. Masses of the eluted compounds were detected with the following settings: capillary 1 kV, source temperature 140° C., desolvation temperature 450° C., and desolvation gas flow 800 l h⁻¹. Argon was used as the collision gas. MS/MS was performed in negative ionization mode according to the observed deprotonated masses. The following settings were used: a capillary spray of 1 kV; cone voltage of 30 eV; collision energy ramp of 15-50 eV.

### Tripple Quad Analysis

Injections were performed on a UPLC (Waters) connected to a Triple Quad detector (TQ-S, Waters) in multiple reaction monitoring (MRM) mode. The chromatographic separation was achieved using a similar column and mobile phase as previously described. A short 7 min method was established using the following multistep gradient program: initial conditions were 57% B raised to 85% B until 4 min, raised to 100% B until 4.2 min, held at 100% B until 6 min, decreased to 67% B until 6.2 min, and held at 67% B until 7 min for re-equilibration of the system. A flow rate of 0.6 ml min⁻¹ was used, the column temperature was 40° C., and the injection volume was 1 μl. The instrument was operated in negative mode with a capillary voltage of 1.5 kV, and a cone voltage of 40 V. Two different transitions were used for CBGA analysis (359.3>191.2, 32 V for quantification; and 359.3>315.4, 21 V for qualification).

### Trichome Isolation

Young leaves were harvested and soaked in ice-cold, distilled water and then abraded using a BeadBeater machine (Biospec Products, Bartlesville, OK). The polycarbonate chamber was filled with 15 g of plant material, and with half the volume with glass beads (0.5 mm diameter), XAD-4 resin (1 g/g plant material), and ethanol 80% to full volume. Leaves were beaten by 2-4 pulses of operation of 1 min each. This procedure was carried out at 4° C., and after each pulse the chamber was allowed to cool on ice. Following abrasion, the contents of the chamber were first filtered through a kitchen mesh strainer and then through a 100 μm nylon mesh to remove the plant material, glass beads, and XAD-4 resin. The residual plant material and beads were scraped from the mesh and rinsed twice with additional ethanol 80% that was also passed through the 100 μm mesh. The presence of enriched glandular trichome secretory cells was checked by visualization in an inverted optical microscope.

### Genome Sequencing and Assembly of Helichrysum

The genome size of Helichrysum was estimated by flow cytometry. Briefly, nuclei were isolated by chopping young leaf tissue of Helichrysum and tomato (used as known reference) in isolation buffer. The samples were stained with propidium iodide, and at least 10,000 nuclei were analyzed in a flow cytometer, and the ratio of G1 peak means between both samples was calculated. High molecular weight DNA was extracted from young frozen leaves and sent for sequencing in the Genome Center of UC Davis. The DNA quality was checked by TapeStation traces and a Qubit fluorimeter (Thermo Fisher). Sequencing was done in a Pacbio Sequel II platform, and a ˜12-kilobase DNA SMRT bell library was prepared according to the manufacturer's protocol. Three different SMRT 8M cells were used, yielding 57.8 Gb of HiFi data (˜44× haploid coverage). In addition to Pacbio HiFi data, 200 M reads of PE 2×150 Illumina Hi-C data were obtained by Phase Genomics. Hifiasm software was used to integrate both Pacbio HiFi and HiC data to produce chromosome-scale and haplotype-resolved assemblies.

Further scaffolding of the primary assembly was performed using the Hi-C data and the SALSA software. Ragta g was used for a final round of ordering using the primary assembly as reference to reach syntenic scaffolds for each haplotype. Visualizations of Hi-C data were performed with Juicer and whole-genome alignments with the pafr package (dwinter.github.io/pafr/). Finally, the assembly was softmasked for repetitive elements using EDTA.

### RNA Sequencing and Genome Annotation of Helichrysum

RNA was extracted from seven different tissues: young leaves, old leaves, florets and receptacles of flowers, stems, roots and trichomes. RNA integrity was checked using a TapeStation instrument. Paired-end Illumina libraries were prepared for five of the tissues and sequenced on Illumina HiSeq 3000 instrument (PE 2×150, ˜40 M reads per sample). Random sequencing errors were corrected using Rcorrector and uncorrectable reads were removed. Adaptor and quality trimming were performed using TrimGalore! with the following parameters: —length 36-q 5—stringency 1-e 0.1 (https://github.com/FelixKrueger/TrimGalore). Ribosomal RNA was filtered by discarding reads mapping to SILVA_132_LSURef and SILVA_138_SSURef non-redundant databases using bowtie2—very-sensitive-local mode. Fastq quality checks on each of the steps were performed using MultiQC. The remaining reads were pooled and used for genome-guided de novo transcriptome assembly using Trinity. The Iso-Seq data were obtained from four of the tissues and processed using isoseq3 and cDNA Cupcake ToFU pipelines (https://github.com/Magdoll/cDNA_Cupcake). Fused and unspliced transcripts were removed, and only polyA positive transcripts were kept for a unique set of high-quality isoforms. Iso-Seq and Trinity transcripts were aligned to the assembly using minimap2 and the BAM files were used in the PASA pipeline to generate RNA-based gene model structures. In addition, the novo gene structures were obtained using the software braker2 and the mentioned BAM files as extrinsic training evidence. Finally, ab initio and RNA-based gene models were combined using EvidenceModeler and a final round of PASA pipeline. Gene functional annotation was performed for the predicted mature transcripts using TransDecoder (github.com/TransDecoder/TransDecoder), which considers HMMER hits against PFAM and BLASTP hits against UniProt databases for similarity retention criteria. Further annotation of protein-coding transcripts was performed by BLASTP searches against curated plant protein databases and GO and KEGG terms were obtained with Triannotate.

UMI-based 3′ RNAseq of three replicates of the seven tissues was obtained similarly as described. Adaptor and quality trimming were performed using TrimGalore! in two steps, including PolyA trimming mode. Reads were mapped to the genome using STAR, UMI-deduplicated using umitools, and counts were obtained with featureCounts. Normalization was performed with the varianceStabilizingTransformation algorithm of DESeq2, and the CEMItools package was used for coexpression analysis (dissimilarity threshold of 0.6, pvalue of 0.1). Genes in modules with expression profiles in concordance with the presence of the metabolites of interest were analyzed. Candidate genes were selected based on functional annotations, and blast hits with known enzymes.

### In Vitro Enzymatic Assays With PTs

HuPT1 (SEQ ID NO: 1), HuPT2 (SEQ ID NO: 2), HuPT3 (SEQ ID NO: 3), HuPTx (SEQ ID NO: 7) genes from H. umbraculigerum and GOT4 gene from Cannabis sativa were separately cloned into pESC-HIS vector. Microsomal preparations from yeast cells transformed with pESC-HIS vectors were performed as described by Jozwiak et al. (2020). PT enzymatic assay was carried out as described previously for CsGOT4 (Luo et al., 2019).

### Kinetic Assays of HuPTs

Microsomes (2 μl) were dissolved in reaction buffer (50 mM Tris-HCl, 10 mM MgCl₂, pH 8.5) and substrate was added [0.5 μM-1.5 mM μM olivetolic acid (Cayman Chemicals), 1 mM geranyl pyrophosphate (GPP, Sigma Aldrich)] to a total volume of 50 μl. Samples were incubated for 15 min at 30° C. Samples were extracted with 100 μl ethanol followed by vortexing and centrifugation. The organic layer was filtered and analyzed via UPLC-qTOF and Triple Quad (TQ) instruments.

### Example 1

Functional Characterization of Prenyltransferases (PTs) from H. umbraculigerum

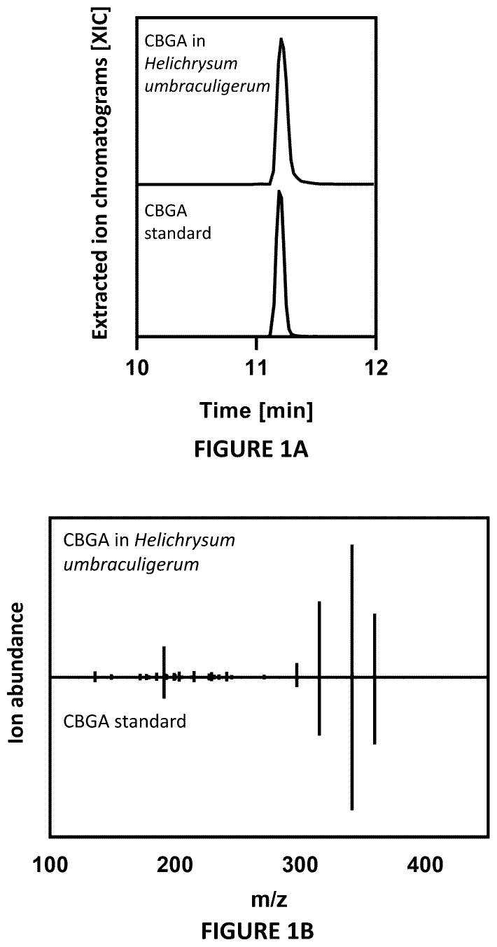

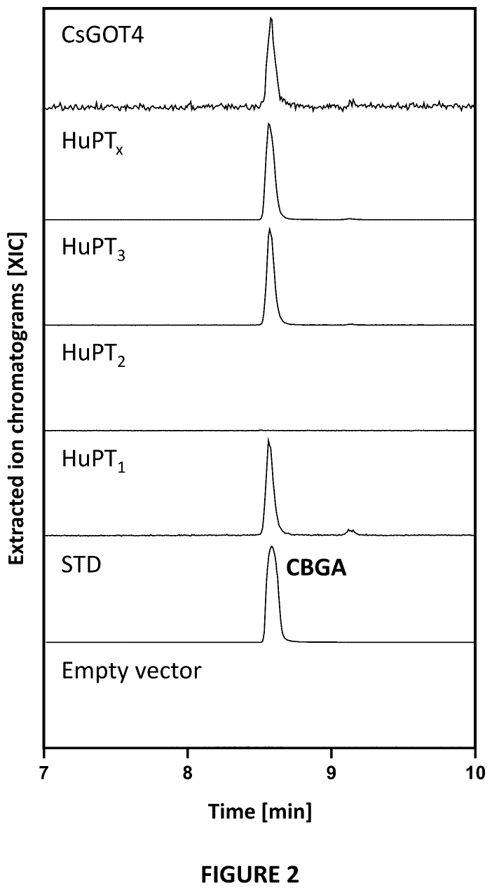

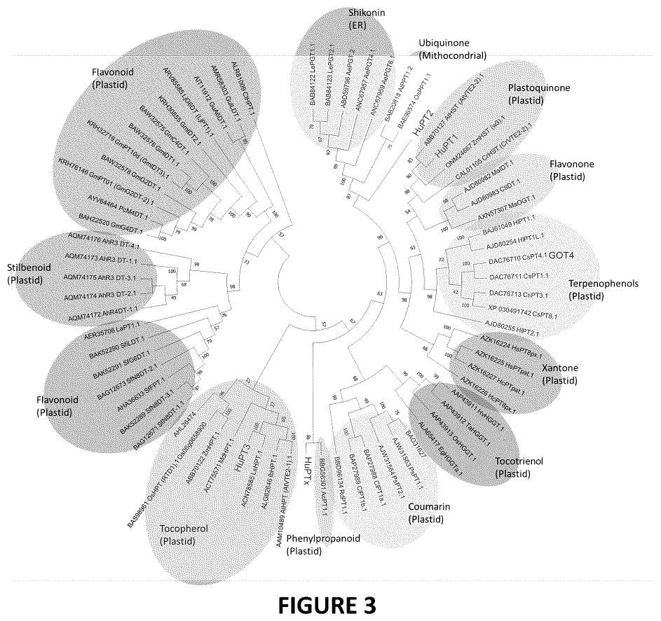

The inventors profiled six tissues (young leaf, old leaf, florets and receptacles of flowers, stems, and roots) from H. umbraculigerum, using UPLC-qTOF, and identified CBGA in all the tissues, excluding roots (FIG. 1). CBGA is the known central precursor to Δ⁹-tetrahydrocannabinolic acid (THCA), cannabidiolic acid (CBDA) and several other cannabinoids. It is produced from olivetolic acid (OA) and geranyl pyrophosphate (GPP) by an enzymatic reaction catalyzed by geranylpyrophosphate:olivetolate geranyltransferase 4 (GOT4). To identify genes in H. umbraculigerum associated with GOT4 like activity, the inventors searched for candidate prenyltransferase genes in Helichrysum transcriptome. Four prenyltransferase like genes, namely, HuPT₁ (SEQ ID NO: 1), HuPT₂ (SEQ ID NO: 2), HuPT₃ (SEQ ID NO: 3) and HuPTx (SEQ ID NO: 7) were selected for further characterization based on their differential expression profile in leaves compared to other tissues. All these PTs shared less than 40% homology with CsGOT4 that is known to partake in cannabinoid biosynthesis. For functional expression in yeast, the inventors removed the N-terminal plastid targeting sequences from all four PTs. Then, each PT candidate expression cassette was introduced into yeast. Furthermore, microsomal fractions were purified from yeast cells expressing candidate PT, and PT activity was examined using OA and GPP as substrates. Purified yeast microsomal fraction containing GOT4 from Cannabis sativa was used with OA and GPP in positive control reaction. Of the four candidates tested, assay with PTI, PT3, and PTx enzymes from H. umbraculigerum showed clear production of CBGA, similarly as also observed in the positive control reaction (FIG. 2). The active HuPTs clustered with plastidial PTs that prenylate diverse substrates, whereas HuPT₂ clustered with mitochondrial PTs (FIG. 3).

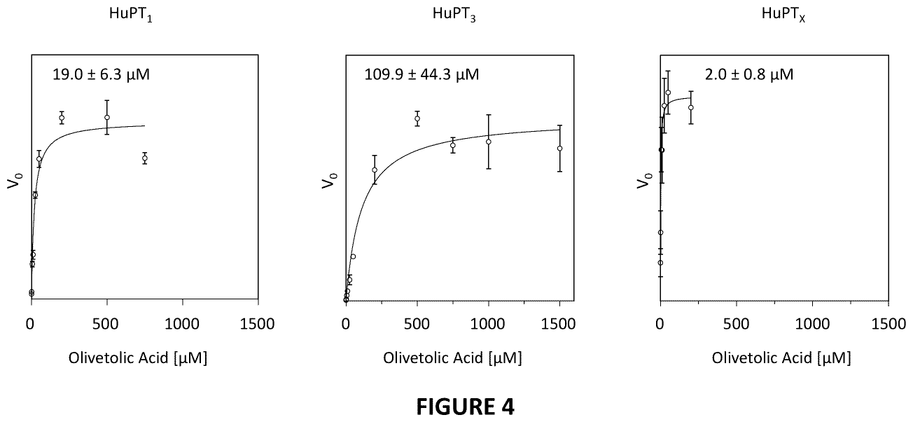

Further, in in vitro assays with purified microsomal fractions containing HuPT₁, HuPT₃ and HuPTx, the inventors confirmed the activity of these enzymes towards CBGA production. Kinetic assays revealed that HuPTx exhibited a very high catalytic activity (Km 2.0±0.8 μM, FIG. 4). The other HuPTs, HuPT₁, and HuPT₃, were also catalytically active (Km 19.0±6.3 μM, and Km 109.9±44.3 μM, respectively). The activity of HuPTx exhibited a similar activity to that of GOT4 from Cannabis sativa (Km CsGOT4=6.7±0.3 μM, Luo et al., 2019). The results show that, HuPTs, such as HuPTx, could therefore be excellent enzymes for the production of cannabinoids in heterologous systems.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

What is claimed is:

1. An isolated DNA molecule comprising a nucleic acid sequence having at least 91% homology to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or any combination thereof.

2. The isolated DNA molecule of claim 1, wherein said nucleic acid sequence having at least 80% homology to any one of SEQ ID Nos.: 1-11 is 950 to 1,750 nucleotides long, and optionally wherein said nucleic acid sequence encodes a protein being a prenyl transferase.

3. (canceled)

4. An artificial nucleic acid molecule, a plasmid or an agrobacterium comprising the isolated DNA molecule of claim 1.

5. (canceled)

6. An isolated protein encoded by the isolated DNA molecule of claim 1.

7. The isolated protein of claim 6, comprising an amino acid sequence with at least 92% homology to SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, or SEQ ID NO: 22, or consisting thereof.

8. (canceled)

9. The isolated protein of claim 6, characterized by being capable of transferring a prenyl group to a substrate molecule, and wherein any one of: said prenyl group is selected from the group consisting of: dimethylallyl diphosphate, geranyl diphosphate, farnesyl diphosphate, and geranylgeranyl diphosphate, said substrate molecule is represented by Formula I: 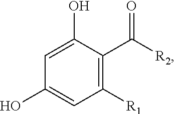
wherein: (i) R₁ is selected from the group consisting of: C1-C8 alkyl, and alpha-unsaturated phenylalkyl carboxylic acid; and R₂ is OH; or (ii) R₁ is OH and R₂ is selected from the group consisting of: C1-C8 alkyl, and alpha-unsaturated phenylalkyl carboxylic acid, said alpha-unsaturated phenylalkyl carboxylic acid comprises cinnamic acid or a derivative thereof, said cinnamic acid derivative is a hydroxylated derivative of cinnamic acid, said hydroxylated derivative of cinnamic acid is coumaric acid, and any combination thereof.

10.-14. (canceled)

15. A transgenic cell comprising the isolated DNA molecule of claim 1.

16. The transgenic cell of claim 15, being any one of: a unicellular organism, a cell of a multicellular organism, and a cell in a culture, and optionally wherein said unicellular organism comprises a fungus or a bacterium, said fungus is a yeast cell, or both.

17.-18. (canceled)

19. An extract derived from the transgenic cell of claim 15, and optionally wherein said extract comprises an isolated protein encoded from said isolated DNA molecule, said, or both.

20. (canceled)

21. A transgenic plant, a transgenic plant tissue or a plant part, comprising, the isolated DNA molecule of claim 1, and optionally wherein said transgenic plant being a Cannabis sativa plant.

22. (canceled)

23. A composition comprising the isolated DNA molecule of claim 1, and an acceptable carrier.

24. A method for synthesizing a compound represented by Formula II: 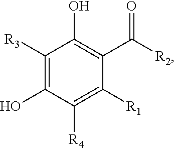
wherein: R₁ is selected from the group consisting of: C1-C8 alkyl, and alpha-unsaturated phenylalkyl carboxylic acid, and R₂ is OH; or (ii) R₁ is OH and R₂ is selected from the group consisting of: C1-C8 alkyl, and alpha-unsaturated phenylalkyl carboxylic acid, wherein R₃ is a prenyl group, and wherein R₄ is hydrogen or a prenyl group, the method comprising:
a. providing a cell comprising an artificial vector comprising a nucleic acid sequence having at least 91% homology to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11; and
b. culturing said cell from step (a) such that a protein encoded by said artificial vector is expressed,
thereby synthesizing the compound represented by Formula II.

25. The method of claim 24, wherein any one of: (1) said protein is characterized by being capable of transferring a prenyl group to a substrate molecule, and optionally wherein said culturing comprises supplementing said cell with an effective amount of said substrate molecule, said substrate molecule is represented by Formula I: 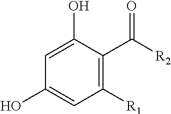
wherein: (i) R₁ is selected from the group consisting of: C1-C8 alkyl, and alpha-unsaturated phenylalkyl carboxylic acid; and R₂ is OH; or (ii) R₁ is OH and R₂ is selected from the group consisting of: C1-C8 alkyl, and alpha-unsaturated phenylalkyl carboxylic acid, or both; (2) said artificial vector is an expression vector; (3) said cell is a prokaryote cell or a eukaryote cell; (4) said cell is a transgenic cell or a cell transfected with the isolated DNA molecule of claim 1; (5) any combination of (1) to (4), and optionally wherein said method further comprises a step preceding step (a), comprising introducing or transfecting said cell with said artificial vector.

26.-31. (canceled)

32. A method for synthesizing a compound represented by Formula II: 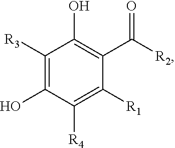
wherein: R₁ is selected from the group consisting of: C1-C8 alkyl, and alpha-unsaturated phenylalkyl carboxylic acid, and R₂ is OH; or (ii) R₁ is OH and R₂ is selected from the group consisting of: C1-C8 alkyl, and alpha-unsaturated phenylalkyl carboxylic acid, wherein R₃ is a prenyl group, and wherein R₄ is hydrogen or a prenyl group, the method comprising contacting a substrate molecule with an effective amount of a protein comprising an amino acid sequence with at least 92% homology to SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, or SEQ ID NO: 22, wherein said substrate molecule is represented by Formula I: 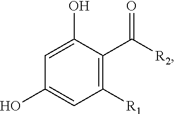
wherein: (i) R₁ is selected from the group consisting of: C1-C8 alkyl, and alpha-unsaturated phenylalkyl carboxylic acid; and R₂ is OH; or (ii) R₁ is OH and R₂ is selected from the group consisting of: C1-C8 alkyl, and alpha-unsaturated phenylalkyl carboxylic acid, thereby synthesizing the compound represented by Formula II, and optionally wherein said contacting is in a cell-free system.

33. (canceled)

34. The method of claim 25, wherein any one of: (i) said substrate molecule is selected from the group consisting of: a resorcinoid precursor, a stilbene acid precursor, an acyl phloroglucinoid precursor, and a chalcone precursor; (ii) wherein said substrate molecule is represented by a formula selected from the group consisting of: 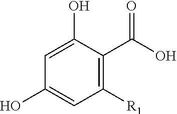
wherein R1 is C1-C8 alkyl, and wherein R2 is alpha-unsaturated phenylalkyl carboxylic acid; (iii) said substrate molecule is selected from the group consisting of:

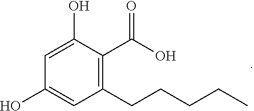 [SMILES: CCCCCc1cc(O)cc(O)c1C(=O)O]

35.-36. (canceled)

37. The method of claim 24, wherein any one of: (i) said compound is selected from the group consisting of: a cannabinoid, an amorfrutin, an acyl phlorogluconoid, and a prenyl chalcone; (ii) said compound is selected from the group consisting of: 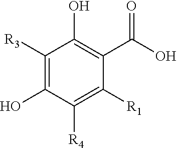
wherein: R1 is C1-C8 alkyl, R2 is an alpha-unsaturated phenylalkyl carboxylic acid, R3 is a prenyl group, and R4 is hydrogen or a prenyl group; (iii) said prenyl group is selected from the group consisting of: dimethylallyl diphosphate, geranyl diphosphate, farnesyl diphosphate, and geranylgeranyl diphosphate; (iv) said alpha-unsaturated phenylalkyl carboxylic acid comprises cinnamic acid or a derivative thereof, and optionally wherein said cinnamic acid derivative is a hydroxylated derivative of cinnamic acid, said hydroxylated derivative of cinnamic acid is coumaric acid, or both; (v) said compound is selected from the group consisting of: 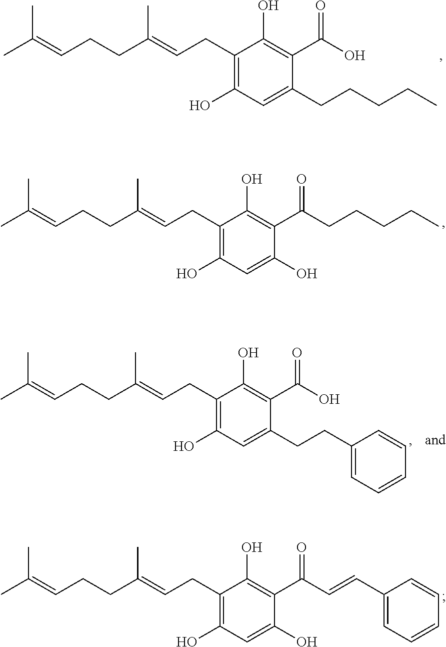
(vi) said compound is 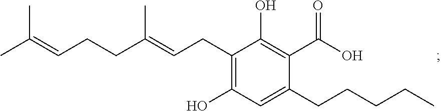 [SMILES: CCCCCc1cc(O)c(C/C=C(\C)CCC=C(C)C)c(O)c1C(=O)O]
and (vii) any combination of (i) to (vi).

38.-44. (canceled)

45. A method for obtaining an extract from a transgenic cell comprising the steps:
a. culturing the transgenic cell of claim 15 in a medium; and
b. extracting said transgenic cell,
thereby obtaining an extract from the transgenic cell, and optionally wherein said method further comprises a step preceding step (b), comprising separating said cultured transgenic cell from said medium.

46. (canceled)

47. An extract of a transgenic cell obtained according to the method of claim 45.

48. A medium or a portion thereof separated from a cultured transgenic cell, obtained according to the method of claim 45.

49. A composition comprising the extract of claim 47, and an acceptable carrier.
